(19) United States
(12) Patent Application Publication
(10) Pub. No.: US 2006/0099169 A1
(43) Pub. Date: May 11, 2006

(54) Toxin binding compositions

(75) Inventors: Dominique Charmot, Campbell, CA (US); Jerry M. Buysse, Los Altos, CA (US); Han Ting Chang, Livermore, CA (US); Michael J. Cope, Berkeley, CA (US); Tony Kwok-Kong Mong, Sunnyvale, CA (US); Elizabeth Goka, San Jose, CA (US)

Correspondence Address: SENNIGER POWERS, ONE METROPOLITAN SQUARE, 16TH FLOOR, ST LOUIS, MO, 63102, US

(73) Assignee: Ilypsa, Inc., Santa Clara, CA (US)

(21) Appl. No.: 11/249,997

(22) Filed: Oct. 13, 2005

Related U.S. Application Data
(63) Continuation-in-part of application No. 10/965,688, filed on Oct. 13, 2004.
(60) Provisional application No. 60/687,272, filed on Jun. 3, 2005.

(51) Int. Cl. A61K 31/785; A61K 31/74

(52) U.S. Cl. 424/78.27; 977/906

(57) ABSTRACT

Methods and compositions for the treatment of toxin-mediated diseases are provided herein. One aspect of the invention is oligosaccharide-based therapeutics that interact with toxins and methods of uses thereof. In one embodiment the oligosaccharide-based therapeutics of the invention comprise polymeric particles with attached oligosaccharide binding moieties. The compositions of the invention can be used in the treatment of toxin-mediated diseases such as antibiotic-associated diarrhea and pseudomembranous colitis, including Clostridium difficile associated diarrhea.

### REFERENCE TO RELATED APPLICATIONS

This application is a continuation-in-part of U.S. Ser. No. 10/965,688 filed Oct. 13, 2004 and an application claiming the benefit under 35 USC 119(e) of U.S. Ser. No. 60/687,272 filed Jun. 3, 2005.

### BACKGROUND OF THE INVENTION

Bacterial exotoxins represent a wide range of secreted bacterial proteins that have evolved a number of mechanisms to alter critical metabolic processes within a susceptible eukaryotic target cell. In general, these toxins act either by damaging host cell membranes or by modifying proteins that are critical to the maintenance of normal physiologic processes in the cell.

Pseudomembranous enterocolitis (PMC) is recognized as a serious, and sometimes lethal, gastrointestinal disease. The gram-positive sporulating bacterium Clostridium difficile is well-established as the primary etiologic agent of PMC and antibiotic-associated colitis (AAC).

Current therapy for PMC or CDAD patients includes discontinuation of implicated antimicrobial or chemotherapy agents, nonspecific supportive measures, and treatment with antibiotics directed against C. difficile. The most common antimicrobial treatment options include vancomycin, metronidazole, teicoplanin, fusidic acid, and bacitracin. Treatment of CDAD with antibiotics is associated with clinical relapse of the disease. Frequency of relapse is reported to be 5-50%, with a 20-30% recurrence rate being the most commonly quoted figure. Relapse occurs with nearly equal frequency regardless of the drug, dose, or duration of primary treatment with any of the antibiotics listed above. The major challenge in therapy is in the management of patients with multiple relapses, where antibiotic control is problematic.

Several approaches for the direct neutralization of C. difficile toxins activity in the intestinal tract have been reported. In the first, multigram quantities of anion exchange resins such as cholestyramine and colestipol have been given orally in combination with antibiotics. This approach has been used to treat mild to moderately ill patients, as well as individuals suffering from CDAD relapses. See Tedesco, F. J. (1982). “Treatment of recurrent antibiotic-associated pseudomembranous colitis.” Am J Gastroenterol 77(4): 220-1; Mogg, G. A., Y. Arabi, et al. (1980). “Therapeutic trials of antibiotic associated colitis.” Scand J Infect Dis Suppl (Suppl 22): 41-5. Treatment with ion exchange resins does not afford specific removal of toxin A and may remove antibiotics intended to act synergistically with the resins to control CDAD; in addition, the large amounts of resin needed to remove toxin A, combined with their unpleasant taste, restrict the use of such approaches.

In view of the above, there is a need for a compound or combination of compounds that would treat the PMC syndrome caused by C. difficile and other diseases caused by toxins.

### SUMMARY OF THE INVENTION

The present invention is directed to compositions and to methods for the treatment of toxin-mediated diseases.

One aspect of the invention is a toxin binding composition comprising a toxin binding moiety, such as a toxin binding oligosaccharide, and a block copolymer (e.g., as a polymeric particle including for example as a block co-polymeric particle) comprising a hydrophobic block and one or more additional polymeric blocks. Preferably, the toxin binding moiety is attached or linked (i.e., covalently bonded directly or indirectly through a linking moiety) to the one or more additional polymeric blocks of the block copolymer.

In a preferred embodiment within this aspect of the invention, the toxin binding composition comprises a toxin binding oligosaccharide, and a block copolymer (e.g., as a polymeric particle including for example as a block co-polymeric particle). The block copolymer (or copolymeric particle) comprises the hydrophobic block and a hydrophilic block, with the toxin binding oligosaccharide being attached or linked to the hydrophilic block of the block copolymer.

In another preferred embodiment within this aspect of the invention, the toxin binding composition comprises a toxin binding moiety and block copolymer (such as a polymeric particle including for example as a block co-polymeric particle). The block copolymer can comprise a hydrophilic block and a hydrophobic block. The hydrophobic block is chemically crosslinked or physically enveloped such that the block copolymer can form a micelle in an aqueous medium. The toxin binding moiety is attached or linked to the hydrophilic block.

Another aspect of the invention is a toxin binding composition comprising a toxin binding moiety and a polymeric nanoparticle, the toxin binding moiety being linked to the nanoparticle and the nanoparticle being substantially not absorbed from the gastrointestinal lumen into gastrointestinal mucosal cells.

Yet another aspect of the invention is comprising a C. difficile toxin binding moiety and a polymeric particle, wherein at least about 90% of C. difficile toxin A is bound by the composition at a concentration ranging from about 0.1 mg/mL to about 20 mg/mL. The C. difficile toxin A being treated with the toxin binding composition in a phosphate buffer solution containing about 5% fetal bovine serum.

A further aspect of the invention is a toxin binding composition with a C. difficile toxin binding oligosaccharide attached or linked to a particle, such as a polymeric particle, with a mole content of the oligosaccharide per unit surface area of the particle being greater than about 0.3 microequivalents/m² or about 1 micromole/m².

A third aspect of the invention is a protein binding composition comprising an oligosaccharide attached or linked to a particle, such as a polymeric particle, with a mole content of the oligosaccharide per unit surface area of the particle being greater than about 0.3 microequivalents/m² or about 1 micromole/m². The oligosaccharide can bind a water soluble protein. Preferably, the particle is not a protein, is not in form of a dendrimer or a liposome, and is not molecularly water soluble. These compositions preferably have a surface area of about 0.5 m²/gm to about 600 m²/gm and additionally or alternatively, a mole content of oligosaccharide per unit weight greater than about 100 micromol per gram of particle.

In some of the embodiments, including embodiments included within any of the first, second or third aspects of the invention, the particles can be co-polymeric particles with a hydrophobic and hydrophilic block, where the toxin binding moiety (e.g., an oligosaccharide) is attached or linked to the hydrophilic block. The block co-polymers can be in the form of micelles with the hydrophobic block forming the core and the hydrophilic block forming the shell. An additional polymer or polymer block, for example, formed from an additional monomer, can be included, for example, to form or to stabilize the hydrophobic core. In a particularly preferred approach, the micelle can comprise an additional polymer or polymer block that chemically crosslinks or that physically envelopes or that otherwise stabilizes the hydrophobic block of the block copolymer. Examples of suitable additional monomers (suitable for forming the additional core-stabilizing polymer(s)) include, but are not limited to, styrene, divinylbenzene, ethylene glycol dimethacrylate, C₁-C₁₂ alcohol esters of acrylic acid, C₁-C₁₂ alcohol esters of methacrylic acid, vinyltoluene, and vinylesters of C₂-C₁₂ carboxylic acids. Preferably the hydrophilic block is a polymer of dimethylacrylamide and the hydorphobic block is a polymer or co-polymer of C₁-C₁₂ alcohol esters of acrylic acid, C₁-C₁₂ alcohol esters of methacrylic acid, styrene, vinyltoluene, and vinylesters of C₂-C₁₂ carboxylic acids. Preferably the oligosaccharide is 8-methoxycarbonyloctyl-α-D-galactopyranosyl-(1,3)-O-β-D-galactopyranosyl-(1,4)-O-β-D-glucopyranoside. In any of the embodiments of the invention, the particles of the invention can be referred to as microparticles. However, even where certain embodiments are referred to as microparticles, such embodiments are not necessarily limited to certain size ranges of particles. Hence, reference to microparticles is generally intended to refer to small sized particles, for example, having an overall diameter of less than about 1 mm or less. In particular, however, reference to microparticles is not intended to exclude particles that are substantially smaller, including having micron scale or nano scale dimensions (e.g, diameters). Particles comprising oligosaccharides such as toxin binding oligosaccharides can be referred to herein as glycoparticles.

Generally, in embodiments of the first, second or third aspects of the invention, the toxin binding moiety can have a binding affinity for a bacterial toxin, such as a bacterial exotoxin. Hence, the toxin binding moiety can have a binding affinity for a secreted bacterial protein that alters a metabolic process within a eukaryotic cell, such as a mammalian cell, including a human cell. The toxin binding moiety can bind or neutralize a toxin that acts on a mucosal surface of a host. In particular, the mucosal surface can be selected from the group consisting of oral, nasal, respiratory, gastrointestinal, urinary, reproductive and auditory mucosal surfaces.

The compositions described herein can be used in the treatment of toxin-mediated disorders. In some embodiments, the compositions are used in the treatment of C. difficile toxin mediated disorders such as diarrhea, pseudomembranous enterocolitis, or antibiotic-associated colitis.

All publications, patents, and patent applications mentioned in this specification are herein incorporated by reference to the same extent as if each individual publication or patent application was specifically and individually indicated to be incorporated by reference.

### BRIEF DESCRIPTION OF THE DRAWINGS

FIGS. 1A and 1B are schematic representations depicting a method of synthesizing a toxin-binding particle (FIG. 1A) and depicting a toxin-binding particle resulting from such method (FIG. 1B).

FIG. 2 is a schematic representation depicting a summary of ELISA and tissue culture assays used to measure bioactivity of toxin molecules treated with micro-particles.

FIG. 3 is a graph illustrating ELISA profile data for four distinct toxin binding microparticle compositions.

FIG. 4 includes four images illustrating cells and showing toxin B protection afforded by SM1-containing microparticles in a VERO cell assay.

FIG. 5 is a graph depicting binding capacities of microparticles for C difficile Toxin A.

FIG. 6 is a graph depicting binding capacities of microparticles C. difficile Toxin B.

FIG. 7 is a graph depicting the percent removal of C. difficile Toxins A and B by microparticles at different concentrations.

FIG. 8 includes five images illustrating cells and showing toxin A protection afforded by a micelle solution comprising diblock copolymer B in a VERO cell assay.

FIGS. 9A and 9B are graphs illustrating ELISA profile data for two distinct toxin binding microparticles for C. difficile toxin A (FIG. 9A) and toxin B (FIG. 9B).

FIGS. 10A through 10C are images illustrating cells and showing untreated VERO cell monolayer (FIG. 10A), VERO cells treated with C. difficile toxin A (FIG. 10B), and VERO cells treated with both C. difficile toxin A and a toxin-binding microparticle (FIG. 10C).

FIGS. 11A through 11C are graphs illustrating the percentage of C. difficile toxin bound by toxin-binding microparticles of the invention in in-vitro competitive assays involving: toxin A as measured against free oligosaccharides (FIG. 11A); toxin B as measured against free oligosaccharides (FIG. 11B); and both toxin A and toxin B as measured against free carbohydrate monomer SM1 (FIG. 11C).

FIG. 12 is a graph illustrating data that summarizes the results of an in-vivo hamster C. difficile challenge study.

### DETAILED DESCRIPTION OF THE INVENTION

Methods and compositions for binding toxins and treating toxin-mediated diseases are provided herein. In preferred embodiments, the compositions comprise of particles functionalized with toxin-binding moieties, and preferably high density toxin-binding moieties, such as certain oligosaccharide sequences, per unit weight or per unit surface area. The toxin-binding moieties, such as oligosaccharides, can be capable of binding toxins, such as bacterial toxins. Preferred compositions are compositions that bind C. difficile toxins, such as toxin A and/or toxin B. Although many of the embodiments described herein are described and discussed in the context of C. difficile toxins, the invention is not limited to the same.

In certain preferred embodiments, the oligosaccharide sequences employed herein which otherwise display modest affinity to C. difficile toxins, showed a very high binding rate once they are presented at a high density on a particle surface. Not wishing to be bound to a particular theory, it is believed that a high density of oligosaccharide moieties attached to the surface produces a polyvalency effect and results in an increase in binding to the toxins. That is, the global affinity of a particle carrying the oligosaccharides is higher than the summed affinity of the individual oligosaccharides. It is believed that once the first binding event has taken place, the second toxin moiety is presented to a second oligosaccharide in a manner that favors binding enthalpically and/or entropically. Preferably, the toxin binding particles of the present invention comprise of a high density of oligosacchrides per surface unit and/or a limited conformation degree at the surface of the particle. These features are believed to enable a higher toxin binding capacity and/or a greater potency for toxin neutralization in conditions such as CDAD.

The particles described herein can be used in the treatment and/or prevention of toxin-mediated diseases, such as C. difficile associated diarrhea.

A preferred embodiment of the invention is a composition for the removal of C. difficile toxin from an intestinal tract contaminated with toxins. Preferably this composition for the removal of the toxin comprises particles whose surface is presented with covalently attached oligosaccharides with a density greater than about 0.3, 0.4, 0.5, 0.6, 0.7, 0.8, 0.9, 1, 1.1, 1.2, 1.3, 1.4, 1.5 or more microequivalents/m². Preferred density range is about 1 microequivalents/m² to about 15 microequivalents/m²; even more preferred is about 3 microequivalents/m² to about 8 microequivalents/m². The oligosaccharide sequences used can be mono, di, tri, tetra saccharides and higher molecular weight oligosaccharides and have a measurable affinity for bacterial toxins. Suitable oligosaccharides can be branched, linear, or dendritic.

Particles

Particles are preferably selected from inorganic materials such as silica, titanium dioxide, diatomite, zeolites, bentonites, and other metal silicates, or organic polymers prepared from styrene, olefinic, acrylic, methacrylic and vinylic monomers, polycondensates, epoxy resin, polyurethanes, polycarbonates, polyamide, polyimides, formaldehyde based resins, crosslinked hydrogels based on polyamine and polyols, semi-natural polymers such as cellulose ether and cellulose ester. Preferably the selected polymers are non toxic, non biodegradable and non-absorbable. The term “polymer” as used herein includes co-polymers. The particle size ranges preferably from a diameter of about 5 nm to about 1000 micron, more preferably in the range from about 50 nm to about 100 microns, even more preferably from about 75 nm to about 10 microns, even more preferably from about 75 nm to about 1 micron, and most preferably from about 100 nm to about 500 nm.

Some of the various embodiments include a polymeric particle. Preferably, the polymeric particle is a copolymer. One of these embodiments is a toxin binding composition comprising a toxin binding moiety and a polymeric nanoparticle, the toxin binding moiety being linked to the nanoparticle and the nanoparticle being substantially not absorbed from the gastrointestinal lumen into gastrointestinal mucosal cells. In this context, a nanoparticle is a particle having an average particle size less than about 1 micron. In a preferred embodiment, the nanoparticle has a particle size range from about 50 nm to about 800 nm, preferably from about 100 nm to about 500 nm. Also, with respect to these embodiments, the toxin binding composition is localized, upon administration to a subject, in the gastrointestinal lumen of the subject, such as an animal, and preferably a mammal, including for example a human as well as other mammals (e.g., mice, rats, rabbits, guinea pigs, hamsters, cats, dogs, porcine, poultry, bovine and horses). The term “gastrointestinal lumen” is used interchangeably herein with the term “lumen,” to refer to the space or cavity within a gastrointestinal tract, which can also be referred to as the gut of the animal. In some embodiments, the toxin binding composition is not absorbed through a gastrointestinal mucosa. “Gastrointestinal mucosa” refers to the layer(s) of cells separating the gastrointestinal lumen from the rest of the body and includes gastric and intestinal mucosa, such as the mucosa of the small intestine. In some embodiments, lumen localization is achieved by efflux into the gastrointestinal lumen upon uptake of the toxin binding composition by a gastrointestinal mucosal cell. A “gastrointestinal mucosal cell” as used herein refers to any cell of the gastrointestinal mucosa, including, for example, an epithelial cell of the gut, such as an intestinal enterocyte, a colonic enterocyte, an apical enterocyte, and the like. Such efflux achieves a net effect of non-absorbedness, as the terms, related terms and grammatical variations, are used herein.

In preferred approaches, the toxin binding composition can be a composition that is substantially not absorbed from the gastrointestinal lumen into gastrointestinal mucosal cells. As such, “not absorbed” as used herein can refer to compositions adapted such that a significant amount, preferably a statistically significant amount, more preferably essentially all of the toxin binding composition, remains in the gastrointestinal lumen. For example, at least about 80% of toxin binding composition remains in the gastrointestinal lumen, at least about 85%, 90%, 95%, or 98% of toxin binding composition remains in the gastrointestinal lumen (in each case based on a statistically relevant data set).

Reciprocally, stated in terms of serum bioavailability, a physiologically insignificant amount of the toxin binding composition is absorbed into the blood serum of the subject following administration to a subject. For example, upon administration of the toxin binding composition to a subject, not more than about 20% of the administered amount of toxin binding composition is in the serum of the subject (e.g., based on detectable serum bioavailability following administration), preferably not more than about 15% of toxin binding composition, and most preferably not more than about 10% of toxin binding composition is in the serum of the subject. In some embodiments, not more than about 5%, not more than about 2%, preferably not more than about 1%, and more preferably not more than about 0.5% is in the serum of the subject (in each case based on a statistically relevant data set).

The term “not absorbed” is used interchangeably herein with the terms “non-absorbed,” “non-absorbedness,” “non-absorption” and its other grammatical variations.

Among various preferred embodiments is a toxin binding composition comprising a C. difficile toxin binding moiety and a polymeric particle. At least about 90% of C. difficile toxin A is bound by the composition at a concentration ranging from about 0.1 mg/mL to about 20 mg/mL. The C. difficile toxin A being treated with the toxin binding composition in a phosphate buffer solution containing about 5% fetal bovine serum. Preferably, the concentration of the toxin binding composition needed to bind about 90% of C. difficile toxin A is from about 0.5 mg/mL to about 10 mg/mL; more preferably, from about 0.8 mg/mL to about 5 mg/mL; even more preferably, from about 1 mg/mL to about 3 mg/mL. In other preferred embodiments, at least about 90% of C. difficile toxin B is bound by the composition at a concentration ranging from about 0.1 mg/mL to about 20 mg/mL. The C. difficile toxin B being treated with the toxin binding composition in a phosphate buffer solution containing about 5% fetal bovine serum. Preferably, the concentration of the toxin binding composition needed to bind about 90% of C. difficile toxin B is from about 0.8 mg/mL to about 10 mg/mL; more preferably, from about 1 mg/mL to about 6 mg/mL.

In some of these various embodiments, the C. difficile toxins A and B are purified. Incubation of the C. difficile toxin A and/or B with toxin binding composition can be carried out for about 2 hours to about 36 hours; preferably, from about 4 hours to about 24 hours; more preferably from about 12 hours to about 18 hours. The incubation typically is carried out at a temperature ranging from about 30° C. to about 40° C.; preferably about 37° C. The amount of toxin bound to the polymeric particle was calculated from determining the amount of free toxin in the supernatant by C. difficile toxin ELISA and subtracting from the amount of C. difficile toxin added to the mixture. The values resulting from the tests are tabulated in Table 8 and described in more detail in Example 8.

The particles can be any suitable shape, preferably spherical, lamellar, or irregular. The most preferred shape is spherical. The particle itself can be microporous, macroporous, mesoporous, or non-porous. If large sized particles are used, it is preferred that these particles are porous so that the surface available for toxin binding is higher. The pore size distribution is preferably selected so as to allow toxin to access the internal surface of the particles. For example, for high molecular weight toxins such as toxin A and B secreted by C. difficile, required pore size is least two times larger than the toxin diameter. For non-porous particles, such as spherical beads, the surface is limited to the outer surface, so preferably the size of the beads is adjusted so that enough surfaces is available to neutralize the toxin load present in the GI at a particular dosage.

In preferred embodiments, the toxin binding moiety (e.g., oligosaccharide) surface density can be greater than about 0.3, 0.4, 0.5, 0.6, 0.7, 0.8, 0.9, 1, 1.1, 1.2, 1.3, 1.4, 1.5 or more micromol/m². For example, the mole content of the toxin binding moiety (e.g., oligosaccharide) per unit surface density of the particle can be greater than about 1 micromol/m², and can range for example from about 1 micromol/m² to about 10 micromol/m², preferably from about 1 micromol/m^{2 to} about 5 micromol/m² and in some embodiments from about 1 micromol/m² to about 3 micromol/m². In certain embodiments, the surface density can be about 2 or about 3 micromol/m². In other preferred embodiments, the toxin binding moiety (e.g., oligosaccharide) surface density can be greater than about 0.5, 0.1 or 1.5 micromol/m². Additionally or alternatively, the particles can have a mole content of toxin binding moiety (e.g., oligosaccharide) per unit weight preferably in the range of about 10 micromol/gm to about 1000 micromol/gm. A preferred toxin binding (e.g., oligosaccharide) mole content per unit weight of particle can range from about 10 micromol/gm to about 500 micromol/gm, or from about 10 micromol/gm to about 200 micromol/gm, or from about 10 micromol/gm to about 100 micromol/gm. In some embodiments, the mole content per unit weight can be about 70 micromol/gm.

The information in Table 1 may be used to guide the choice of particle size and porosity for a given oligosaccharide content.

TABLE 1

| | Surface | Mole per | Required | Particle |
|---|---|---|---|---|
| | density | weight | surface for | size |
| | (μmole/m²) | (μmole/gm) | binding (m²/gm) | (micron) |
| Porous | 10 | 10 | 1 | |
| | 10 | 50 | 5 | |
| | 10 | 150 | 15 | |
| Non porous | 15 | 10 | | |
| spheres | | | | |
| | 15 | 100 | | 0.9 |
| | 15 | 300 | | 0.3 |
| | 15 | 500 | | 0.18 |

In some embodiments, the particles are liposomes or vesicles formed from association of phospholipids, as well as other similar type of macromolecular assemblies such as block polymer micelles. In other embodiments, the particles are dendritic structures such as those known in the art, e.g., see Grayson S. M. et al. Chemical Reviews, 2001, 101: 3819-3867; and Bosman A. W. et al, Chemical Reviews, 1999, 99; 1665-1688, incorporated herein by reference.

In one embodiment, the toxin binding composition comprises of at least two particles, the two particles being attached to each other and the oligosaccharide being attached to one of the particles. Preferably, one of the particles is a co-polymer. In certain embodiments, the second particle is a latex particle, silica particle, methyloxide nanoparticle, hydrophobic polymer, colloidal polymer, or is made of other suitable materials described herein.

Particle Formation

Depending upon the size and morphology of the particle selected as the oligosaccharide carrier, various synthetic procedures can be used. For instance, silica particle with non porous, spherical shape are conveniently prepared using sol-gel process, in particular the Stober process whereby a silicon alkoxide is co-hydrolyzed with ammonia (Stober et al, Journal of Colloid and Interface Science, 1968, 26, 62). Other sol-gel processes using either organometallic or metallic salts are also well known to produce metal oxides nanoparticles. Aerosol and jetting processes are also common to prepare well controlled inorganic and organic material powder with characteristics of size and porosity well suited to the present invention. Organic polymeric beads can be prepared by polymerization in dispersed media, such as suspension, microsuspension, emulsion, miniemulsion, microemulsion polymerizations methods. When porous particles are used, suspension polymerization processes are preferred wherein mixtures of free radical polymerizable monomers including multifunctional monomers are emulsified in an aqueous phase with dispersing agents, said monomer phase also includes a variety of diluent and porogen solvents. The latter solvents control the micro/macro/meso porosity of the formed particles. Mono-sized particles are prepared by multi-step seeded suspension polymerization or alternatively using membrane emulsification or jetting processes. Generally, monomers that may be co-polymerized to prepare such polymer particles include at least one monomer selected from the group consisting of styrene, divinylbenzene (all isomers) substituted styrene, alkyl acrylate, substituted alkyl acrylate, alkyl methacrylate, substituted alkyl methacrylate, acrylonitrile, ethyleneglycol dimethacrylate, methacrylonitrile, acrylamide, methacrylamide, N-alkylacrylamide, N-alkylmethacrylamide, N,N-dialkylacrylamide, N,N-dialkylmethacrylamide, isoprene, butadiene, ethylene, vinyl acetate, N-vinyl amide, maleic acid derivatives, vinyl ether, allyle, methallyl monomers and combinations thereof. Functionalized versions of these monomers may also be used. Specific monomers or comonomers that may be used in this invention include methyl methacrylate, ethyl methacrylate, propyl methacrylate (all isomers), butyl methacrylate (all isomers), 2-ethylhexyl methacrylate, isobornyl methacrylate, methacrylic acid, benzyl methacrylate, phenyl methacrylate, methacrylonitrile, α-methylstyrene, methyl acrylate, ethyl acrylate, propyl acrylate (all isomers), butyl acrylate (all isomers), 2-ethylhexyl acrylate, isobornyl acrylate, acrylic acid, benzyl acrylate, phenyl acrylate, acrylonitrile, styrene, glycidyl methacrylate, 2-hydroxyethyl methacrylate, hydroxypropyl methacrylate (all isomers), hydroxybutyl methacrylate (all isomers), N,N-dimethylaminoethyl methacrylate, N,N-diethylaminoethyl methacrylate, triethyleneglycol methacrylate, itaconic anhydride, itaconic acid, glycidyl acrylate, 2-hydroxyethyl acrylate, hydroxypropyl acrylate (all isomers), hydroxybutyl acrylate (all isomers), N,N-dimethylaminoethyl acrylate, N,N-diethylaminoethyl acrylate, triethyleneglycol acrylate, methacrylamide, N-methylacrylamide, N,N-dimethylacrylamide, N-tert-butylmethacrylamide, N-n-butylmethacrylamide, N-methylolmethacrylamide, N-ethylolmethacrylamide, N-tert-butylacrylamide, N-n-butylacrylamide, N-methylolacrylamide, N-ethylolacrylamide, 4-acryloylmorpholine, vinyl benzoic acid (all isomers), diethylaminostyrene (all isomers), α-methylvinyl benzoic acid (all isomers), diethylamino α-methylstyrene (all isomers), p-vinylbenzene sulfonic acid, p-vinylbenzene sulfonic sodium salt, trimethoxysilylpropyl methacrylate, triethoxysilylpropyl methacrylate, tributoxysilylpropyl methacrylate, dimethoxymethylsilylpropyl methacrylate, diethoxymethylsilylpropyl methacrylate, dibutoxymethylsilylpropyl methacrylate, diisopropoxymethylsilylpropyl methacrylate, dimethoxysilylpropyl methacrylate, diethoxysilylpropyl methacrylate, dibutoxysilylpropyl methacrylate, diisopropoxysilylpropyl methacrylate, trimethoxysilylpropyl acrylate, triethoxysilylpropyl acrylate, tributoxysilylpropyl acrylate, dimethoxymethylsilylpropyl acrylate, diethoxymethylsilylpropyl acrylate, dibutoxymethylsilylpropyl acrylate, diisopropoxymethylsilylpropyl acrylate, dimethoxysilylpropyl acrylate, diethoxysilylpropyl acrylate, dibutoxysilylpropyl acrylate, diisopropoxysilylpropyl acrylate, maleic anhydride, N-phenylmaleimide, N-butylmaleimide, N-vinylformamide, N-vinyl acetamide, allylamine, methallylamine, allylalcohol, methyl-vinylether, ethylvinylether, butylvinyltether, butadiene, isoprene, chloroprene, ethylene, vinyl acetate and combinations thereof.

The oligosaccharide moiety can be attached on the particle surface following various routes, for instance by first functionalizing the oligosaccharide sequence with an amine reactive end-group preferably located on the reducing end of the sugar group and further reacting the amine reactive functional saccharide to an amine-functionalized particle, such as a thioisocyanato group. A variant of this approach is to attach the amine functional group on the oligosaccharide and have it react with particles functionalized with an electrophile, such an epoxide group.

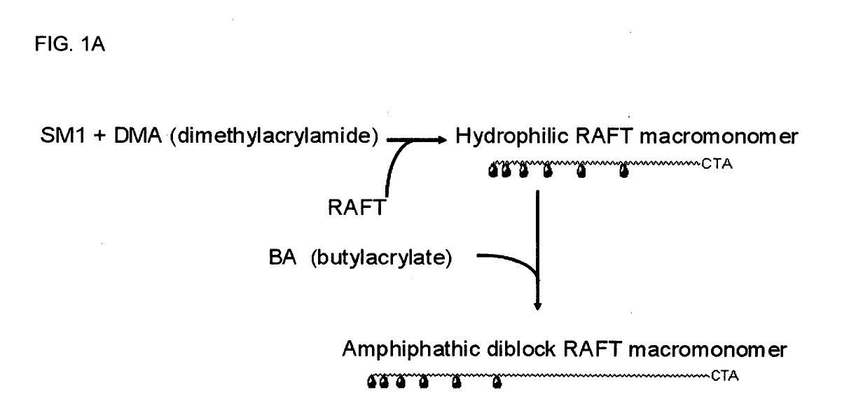

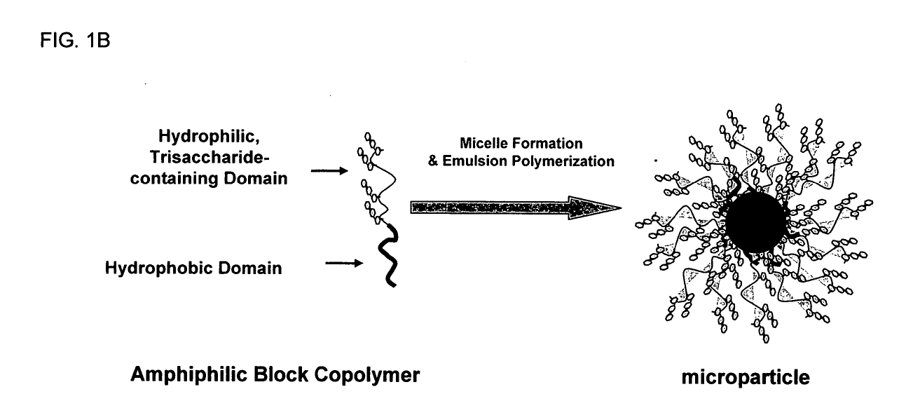

In another method, a polymerizable moiety is first attached to the oligosaccharide and copolymerizing this oligosaccharide functional monomer with particle-forming monomer in an emulsion polymerization process. A variant of this general process and preferred embodiment is to first polymerize the oligosaccharide functional monomer with a second co-monomer using a living polymerization technique, to form a first hydrophilic block; secondly, using this hydrophilic block to further grow a second hydrophobic block, to form a diblock copolymer; and thirdly, dispersing the block copolymers in an aqueous media. Block copolymer synthesis can be performed by a number of living polymerization techniques such as anionic, cationic, group transfer polymerization and controlled free radical polymerization. The latter techniques include nitroxide mediated polymerization, atom transfer radical polymerization (ATRP), and reversible addition fragmentation transfer (RAFT); the latter technique being preferred. RAFT techniques employ chain transfer agent (CTA) selected from dithioesters, dithiocarbamates, dithiocarbonate, or dithiocarbazates. A schematic view of the approach is given in FIGS. 1A and 1B. The amphiphilic block copolymers spontaneously assemble into micelles, comprising a core of the collapsed hydrophobic blocks and a shell of the oligosaccharide functional hydrophilic blocks. In another preferred embodiment, the hydrophobic core of the block copolymer micelles is further crosslinked by polymerizing an additional third monomer or “core-filling” monomer. This core-filling monomer is preferably a hydrophobic monomer, a multifunctional monomer, or a combination thereof. The weight ratio of the core-filling monomer to the block copolymer is typically comprised between about 0.1 to about 100, preferably between about 0.5 to about 10.

The block copolymers have a molecular weight in a range of about 2000 to about 200,000, preferably about 500 to about 200,000, more preferably about 10,000 to about 100,000, most preferably about 20,000 to about 50,000; a ratio of hydrophilic to hydrophobic comprised between about 9:1 to about 1:9, preferably about 3:1 to about 1:3, more preferably about 2:1 to about 1:2, even more preferably about 1.1:1, and most preferably about 1.5:1; and an oligosaccharide mole fraction in the hydrophilic block in the range of about 2 mole percent to about 100 mole percent, preferably about 5 mole percent to about 50 mole percent.

The oligosaccharides can be attached to a polymeric particle via various methods, by the use of a dendritic spacer. For example, methods of using dendritic spacers are described in Lundquist and Toone, The Cluster Glycoside Effect, Chem. Rev., 2002, 102, 555-578.

In certain preferred embodiments, the oligosaccharides are anchored on a solid surface at a high local density. The control in the sugar density can be achieved by the synthetic procedures just described. Process variables include the sugar content in the block copolymer, the ratio of the sugar-containing block to the hydrophobic block, and the ratio of block copolymer to core-filling monomer. The sugar surface density can be first approximated from the particle surface and the sugar content in the recipe. The particle surface can be computed from the particle size as measured by electron microscopy, dynamic light scattering or Fraunhoffer light diffraction methods. Alternatively the mole content of oligosaccharide can be determined by knowing the initial sugar concentration. Preferably, the oligosaccharide surface density is greater than about 1 μmole/m², preferably greater than about 5 μmole/m² and most preferably greater than about 10 μmole/m². Optimal density range is determined by the binding capacity of toxin as measured by standard biochemistry and cell biology procedures such as those described below.

In another aspect of the invention, methods are provided for the synthesis of the trisaccharide Gal(α1-3)Gal(β1-4)Glc with a methyl ester handle for linker modifications. An example of such modification includes the introduction of a diamine group to serve as a linker for the addition of a variety of polymer backbone structures. In another aspect of the invention, methods for the production of the polymer backbones and trisaccharide-linker-polymer compositions are described, based on free radical polymerization techniques. Such techniques include direct polymerization of polymerizable sugar monomers using sugar-derived acrylate, methacrylate, styrenic, and vinyl monomers; additional techniques include post-modifying the complete polymer with sugar moieties, using nucleophilic amine sugars to react with copolymers containing epoxide or activated ester groups. Characteristics of the trisaccharide-linker-polymer that can be altered to produce a high affinity toxin A binder include polymer size, oligosaccharide density within the polymer, balance of hydrophobicity/hydrophilicity in the finished polymer, and architecture/morphology of the monomer subunits (i.e., linear, block, star, graft, and gel).

Toxin-Binding Oligosaccharides

Examples of suitable oligosaccharides that can be used in the compositions described herein include oligosaccharides that bind toxin A and/or toxin B. Suitable oligosaccharides include C. difficile toxin binding oligosaccharides such as βGlc; αGlc(1-2)βGal; αGlc(1-4)βGlc (maltose); βGlc(1-4)βGlc (cellobiose); αGlc(1-6)αGlc(1-6)βGlc (somaltose); αGlc(1-6)βGlc (isosomaltose); βGlcNAc(1-4)βGlcNAc (chitobiose). Other suitable C. difficile toxin binding oligosaccharides include:

| | |
|---|---|
| αGal(1-3)βGal(1-4)βGlc | |
| αGal(1-3)βGal(1-4)βGlcNAc | |
| βGal(1-4)βGlcNAc | (human blood group antigen X) |

(1-3)
αFuc

| | |
|---|---|
| βGal(1-4)βGlcNAc | (human blood group antigen Y) |

| | |
|---|---|
| (1-2) | (1-3) |
| αFuc | αFuc |

| | |
|---|---|
| βGal(1-4)βGlcNAc | (human blood group antigen I) |

(1-6)
βGal
(1-3)

βGal(1-4)βGlcNAc

Suitable oligosaccharides for cholera toxin include Gal(β1,3)GalNAc(β1,4)(NeuAc(α2,3))Gal(β1,4)Glc(β)-ceramide; NeuAc(α2,3)Gal(β1,3)GalNAc(β)(NeuAc(α2,3)Gal(β1,4)Glc(β)-ceramide, Gal(β)GalNAc(β1,4)(NeuAc(α2,8)NeuAc(α2,3)Gal(β1,4)Glc(β)-ceramide, GalNAc(β1,4)-Gal(β1,3)GalNAc(β1,4)((NeuAc(α2,3))Gal(β1,4)Glc(β)-ceramide, and Fuc(α1,2)Gal(β1,3)-GalNAc(β1,4)((NeuAc(α2,3))Gal(β1,4)Glc(β)-ceramide.

An example of oligosaccharide for heat-labile toxin is GM1. Suitable oligosaccharides for tetanus toxin are Gal(β1,3)GalNAc(β1,4)((NeuAc(α2,8))NeuAc(2,3)Gal(β1,4)Glc(β)-ceramide; NeuAc(α2,3)Gal(β1,3)GalNAc(β1,4)((NeuAc(α2,8))NeuAc(α2,3)-Gal(β1,4)Glc(β)-ceramide, and NeuAc(α2,8)NeuAc(α2,3)Gal(β1,3)GalNAc(β1,4)(NeuAc(α2,8)-NeuAc(α2,3)Gal(β1,4)Glc(β)-ceramide.

A suitable oligosaccharide for botulinum toxin A and E is NeuAc(α2,8)NeuAc(α2,3)Gal(β1,3)GalNAc(β1,4)(NeuAc(α2,8)) NeuAc(α2,3)-Gal(β1,4)Glc(β)-ceramide; for botulinum toxin B, C, and F is NeuAc(α2,3)Gal(β1,3)GalNAc(β1,4)(NeuAc(α2,8))NeuAc(α2,3) Gal(β1,4)Glc(β)-ceramide; and for botulinum toxin B is Gal(β)-ceramide.

A suitable oligosaccharide for delta toxin is GalNAc(β1,4)(NeuAc(α2,3))Gal(β1,4)Glc(β)-ceramide; for toxin A is Gal(α1,3)Gal(β1,4)GlcNAc(β2,3)Gal(β1,4)Glc(β)-ceramide; for shiga-like toxin (SLT)-I and SLT-II/IIc is Gal(α1,4)Gal(β) (P1 disaccharide), Gal(α1,4)Gal(β1,4)GlcNAc(β) (P1 trisaccharide), or Gal(α1,4)Gal(β1,4)Glc(β) (Pk trisaccharide); for shiga toxin is Gal(α1,4)Gal(β)-ceramide; for vero toxin is Gal(α1,4)Gal(β1,4)Glc(β)-ceramide; for pertussis toxin is NeuAc(α2,6)Gal; and for dysenteriae toxin is GlcNAc(β1).

One aspect of the invention is a protein binding composition comprising an oligosaccharide attached to a particle, wherein the mole content of the oligosaccharide per surface area of the particle is greater than about 0.3, 0.4, 0.5, 0.6, 0.7, 0.8, 0.9, 1 or more microequivalents/m², the oligosaccharide binds a soluble protein, and the particle is not a protein, is not in form of a dendrimer or a liposome, and is not molecularly water soluble. Another aspect of the invention is a protein binding composition comprising an oligosaccharide attached to a particle, wherein the mole content of the oligosaccharide per surface area of the particle is greater than about 0.3, 0.4, 0.5, 0.6, 0.7, 0.8, 0.9, 1 or more microequivalents/m², the oligosaccharide binds a soluble protein, and the particle is not a protein, or carbon nanotube and is not in form of a dendrimer or a liposome, and is not molecularly water soluble. Examples of such particles include lipids, phospholipids and other particles described herein.

Methods of Treatment

In some embodiments, the compositions and methods of the present invention are employed to bind and neutralize toxins. The compositions described herein may bind and/or neutralize all or a portion of the toxins. For example, the toxin may act on mucosal surfaces of the host, including the oral mucosa and gastrointestinal tract, the nasal and respiratory tract, urinary and reproductive tracts, and the auditory canals. Also included are compositions and methods of the invention for use in wounds. Toxins that have a mode of action that inactivates or disrupts the function of cell surface targets are included, with examples found in the family of superantigen toxins elaborated by S. aureus and S. pyogenes, cell permeabilizing toxins such as streptolysin, perfringolysin, alpha-toxin, leukotoxin, aerolysin, delta hemolysin, and the various hemolysins encoded by E. coli pathovars, and toxins that block adhesin function such as Bacteroides fragilis enterotoxin (non-LPS). The invention can also be employed against toxins that bind to the target cell surface, are translocated into the cytoplasm, and disrupt or inactivate intracellular targets. Within this group are included: (i) protein synthesis inhibitors such as diphtheria toxin, P. aeruginosa exotoxin A, and Shiga toxin; (ii) signal transduction inhibitors including anthrax toxin, pertussis toxin and pertussis adenylate cyclase toxin, cholera toxin and related heat labile toxins such as E. coli LT toxin, cytolethal distending toxins produced by H. ducreyi, E. coli, Shigella, and Campylobacter, C. perfringens alpha toxin, C. difficile toxins A and B, and cytotoxic necrotizing factors of E. coli and Bordetella species; and (iii) intracellular trafficking and cytoskeleton toxins, including H. pylori vacuolating toxin, tetanus toxin, the mucosal transport of botulinum toxin, and C2 C botulinum toxin.

The compositions and methods provided herein are employed for the treatment and/or prevention of toxin-mediated diseases. Such toxins can include bacterial toxins and other toxic polypeptides such as, but not limited to, virus particles, prions, antibodies, adhesins, lectins, selectins, signaling peptides, hormones, particularly hormones involve in the immune system response and/or autoimmune diseases, and other molecules that have adverse effects in the GI tract.

The compositions and methods described herein can be employed against bacterial toxins that act at the surface of the target cell and toxins that act on intracellular targets of the susceptible cell. Common examples of the first group include the toxins of S. aureus and S. pyogenes, and pore-forming toxins secreted by a number of gram-positive and gram-negative bacteria including S. aureus, S. pyogenes, C. perfringens, L. monocytogenes, E. coli, A. hydrophila and others. Within the intracellular-acting toxins, examples of toxins which enter the target cell by a receptor-mediated mechanism include P. aeruginosa exotoxin A, S. dysenteriae shiga toxin, V. cholerae cholera toxin, E. coli labile toxin, H. pylori vacuolating toxin, C. botulinum neurotoxin, and C. difficile toxins A and B, along with many other examples. A second group of intracellular-acting toxins gain entry through the direct injection of the toxin into the target cell, common examples of such type III and type IV secreted toxins include the Yop proteins of Y. spp., pertussis toxin of B. pertussis, and the CagA protein of H. pylori. Several bacterial toxins act on cells of the host mucosal surfaces. Among these examples are V. cholerae cholera toxin, E. coli heat labile toxin, S. dysenteriae (including EHEC and EPEC variants) shiga toxin, C. difficile toxin A, B. pertussis pertussis toxin, and the superantigen toxins encoded by S. aureus and S. pyogenes.

Toxigenic strains of C. difficile produce two exotoxins that are responsible for CDAD and the PMC syndrome (Lyerly, D. M., H. C. Krivan, et al. (1988). “Clostridium difficile: its disease and toxins.” Clin Microbiol Rev 1(1): 1-18). Toxin A (CdtA, 308 kDa) is an enterotoxin that causes fluid secretion in animal models and ileal explants and is generally accepted as the primary toxin responsible for producing clinical symptoms (Triadafilopoulos, G., C. Pothoulakis, et al. (1987). “Differential effects of Clostridium difficile toxins A and B on rabbit ileum.” Gastroenterology 93(2): 273-9). Toxin B (CdtB, 279 kDa) is a cytotoxin, as defined by the profound cytopathic effects of the toxin on cultured cells, and its relative lack of enterotoxicity in animal models. By the measure of cytopathic effects alone, toxin B is ˜100-1000 times more toxic than toxin A (Triadafilopoulos, G., C. Pothoulakis, et al. (1987). “Differential effects of Clostridium difficile toxins A and B on rabbit ileum.” Gastroenterology 93(2): 273-9; Lima, A. A., D. M. Lyerly, et al. (1988). “Effects of Clostridium difficile toxins A and B in rabbit small and large intestine in vivo and on cultured cells in vitro.” Infect Immun 56(3): 582-8; Riegler, M., R. Sedivy, et al. (1995). “Clostridium difficile toxin B is more potent than toxin A in damaging human colonic epithelium in vitro.” J Clin Invest 95(5): 2004-11; Chaves-Olarte, E., M. Weidmann, et al. (1997). “Toxins A and B from Clostridium difficile differ with respect to enzymatic potencies, cellular substrate specificities, and surface binding to cultured cells.” J Clin Invest 100(7): 1734-41; Stubbe, H., J. Berdoz, et al. (2000). “Polymeric IgA is superior to monomeric IgA and IgG carrying the same variable domain in preventing Clostridium difficile toxin A damaging of T84 monolayers.” J Immunol 164(4): 1952-60).

The compositions and methods described herein may treat and/or prevent C. difficile toxin-mediated conditions by affecting the toxins inactivation of Rho GTPases by monoglucosylation of a threonine residue involved in the binding of GTP. Glucosylation of Rho GTPases blocks interaction of these signaling molecules with effector proteins that regulate the actin cytoskeleton. In addition, inactivation of Rho GTPases can disrupt the control of secretion processes in the cells, endocytosis, protein synthesis, cell cycle progression, and a number of other fundamental cell “housekeeping” functions. Preferably, the toxin binding compositions inhibit the binding of the C. difficile toxins to host cell surface receptors.

Toxin A binds to glycoconjugates (O-linked, N-linked, or glycosphingolipids) that contain Gal(α1-3)Gal(β1-4)Glc and/or the minimal disaccharide unit Gal(β1-4)Glc comprising the type 2 core (Castagliuolo, I., J. T. LaMont, et al. (1996). “A Receptor Decoy Inhibits the Enterotoxic Effects of Clostridium difficile Toxin A in Rat Ileum.” Gastroenterology 111: 433-8; U.S. Pat. No. 5,484,773; and U.S. Pat. No. 5,635,606). A consensus receptor structure for toxin A has been identified in a variety of nonhuman mammalian cells, but the Gal(α1-3)Gal(β1-4)Glc structure is not naturally found in human tissues. Preferably, the oligosacchride sequences used in the particles of the present invention prevent or inhibit binding of toxin A to these glycoconjugates.

In addition to the treatment of disorders mediated by bacterial toxins, the compositions described herein can be used in other pathological interactions that involve protein-carbohydrate recognition events such as infectious cycles of bacteria, viruses, mycoplasma, and parasites.

In a further aspect of the invention, a method is provided for the treatment of diarrhea mediated by C. difficile toxin A and toxin B, which method comprises administering to a subject suffering CDAD an effective amount of a composition comprising of the trisaccharide Gal(α1-3)Gal(β1-4)Glc linked to a polymer support, wherein said oligosaccharide sequence binds toxin A and removes toxin A from the lumen of the infected gastrointestinal tract. In a similar manner, the composition can bind and remove toxin B, preventing the cytotoxic action of the protein on intestinal epithelial cells. The polymer composition is formulated in an acceptable pharmaceutical carrier, wherein said composition is capable of being eliminated from the gastrointestinal tract.

In another aspect of the invention, the composition consisting of the trisaccharide Gal(α1-3)Gal(β1-4)Glc linked to a polymer support is delivered along with an antibiotic treatment for CDAD, typically consisting of metronidazole (Flagyl) or oral vancomycin; the combination treatment can be provided as separate formulations or in a fixed combination of the agents.

In the present invention, the compositions can be co-administered with other active pharmaceutical agents. This co-administration can include simultaneous administration of the two agents in the same dosage form, simultaneous administration in separate dosage forms, and separate administration. For example, for the treatment of CDAD, the compositions can be co-administered with drugs that cause the CDAD, such as certain antibiotics. The drug being co-administered can be formulated together in the same dosage form and administered simultaneously. Alternatively, they can be simultaneously administered, wherein both the agents are present in separate formulations. In another alternative, the drugs are administered separately. In the separate administration protocol, the drugs may be administered a few minutes apart, or a few hours apart, or a few days apart.

In yet another method, the toxin binding compositions of the invention are coadministered with an effective amount of an antibiotic. The toxin binding compositions can be administered prior to, simultaneous with, or subsequent to the administration of an effective amount of an antibiotic. The dosage and treatment regimen for various antibiotics are well known in the art. In one embodiment, the antibiotic is selected from the group consisting of metronidazole, vancomycin, and combinations thereof. Alternatively, the antibiotic can be selected from the group consisting of teicoplanin, fusidic acid, bacitracin, carbencillim, ampicillin, cloxacillin, oxacillin, pieracillin, cefaclor, cefamandole, cefazolin, cefoperazone, ceftaxime, cefoxitin, ceftazidime, ceftriazone, imipenem, meropenem, nalidixic acid, tetracyclines, gentamicin, paromomycin, and combinations thereof. In a further method, the subject is treated with toxin binding composition and an antibiotic selected from the group consisting of metronidazole, vancomycin, and combinations thereof and, if necessary, subsequently treated with a toxin binding composition and an antibiotic selected from the group consisting of carbencillim, ampicillin, cloxacillin, oxacillin, pieracillin, cefaclor, cefamandole, cefazolin, cefoperazone, ceftaxime, cefoxitin, ceftazidime, ceftriazone, imipenem, meropenem, nalidixic acid, tetracyclines, gentamicin, paromomycin, and combinations thereof.

The term “treating” as used herein includes achieving a therapeutic benefit and/or a prophylactic benefit. By therapeutic benefit is meant eradication, amelioration, or prevention of the underlying disorder being treated. For example, in a pseudomembranous enterocolitis (PMC) patient, therapeutic benefit includes eradication or amelioration of the underlying pseudomembranous exudative plaques attached to the mucosal surface of the intestinal tract. Also, a therapeutic benefit is achieved with the eradication, amelioration, or prevention of one or more of the physiological symptoms associated with the underlying disorder such that an improvement is observed in the patient, notwithstanding that the patient may still be afflicted with the underlying disorder. For example, administration of a C. difficile toxin binding composition to a patient suffering from PMC provides therapeutic benefit not only when the patient's diarrhea is decreased, but also when an improvement is observed in the patient with respect to other disorders that accompany PMC. Examples of prophylactic benefit include when the compositions described herein are administered to a patient at risk of developing PMC or to a patient reporting one or more of the physiological symptoms of PMC, even though a diagnosis of PMC may not have been made. The compositions are also suitable for use in the prevention of reoccurrences of toxin-mediated diseases.

The pharmaceutical compositions of the present invention include compositions wherein the polymers are present in an effective amount, i.e., in an amount effective to achieve therapeutic or prophylactic benefit. The actual amount effective for a particular application will depend on the patient (e.g., age, weight, etc.), the condition being treated, and the route of administration. Determination of an effective amount is well within the capabilities of those skilled in the art, especially in light of the disclosure herein.

The effective amount for use in humans can be determined from animal models. For example, a dose for humans can be formulated to achieve gastrointestinal concentrations that have been found to be effective in animals.

The dosages of the polymers in animals will depend on the disease being, treated, the route of administration, and the physical characteristics of the patient being treated. Dosage levels of the polymers for therapeutic and/or prophylactic uses can be from about about 0.5 gm/day to about 30 gm/day. It is preferred that these polymers are administered along with meals. The compositions may be administered one time a day, two times a day, or three times a day. Most preferred dose is about 15 gm/day or less. A preferred dose range is about 5 gm/day to about 20 gm/day, more preferred is about 5 gm/day to about 15 gm/day, even more preferred is about 10 gm/day to about 20 gm/day, and most preferred is about 10 gm/day to about 15 gm/day. Another preferred dose is about 1 gm/day to about 5 gm/day.

The polymeric compositions described herein can be used in combination with other suitable active agents. For example, in the treatment of PMC or CDAD, the polymeric compositions may be used in combination with antibiotics such as vancomycin, metronidazole, teicoplanin, fusidic acid, and bacitracin. Other combination therapies can include passive immune therapy using anti-toxin A immune globulin or orally-administered bovine anti-toxin A immunoglobulin, toxin A toxoid vaccines, and an oral, non-absorbable polymeric toxin binder based on soluble polystyrene sulfonate resin.

The compositions described herein can be used in combination with anion exchange resins such as cholestyramine and colestipol. Other suitable polymers which can be used in combination are described in U.S. Pat. Nos. 6,007,803; 6,034,129 and 6,290,947 which describe suitable polymers with cationic groups and hydrophobic groups and U.S. Pat. Nos. 6,270,755; 6,419,914; 6,517,827; 6,890,523; and U.S. patent application 2005/0214246 which elate to polymers having anionic groups.

In another method, the linear toxin A binding epitope Gal(α1-3)Gal(β1-4)Glc, and various derivatives, was attached to a solid, inert support to provide an insoluble material capable of binding and neutralizing toxin A (SYNSORB) (Heerze, Armstrong 1996). The oligosaccharide sequence provides a specific binding site for toxin A removal and this receptor mimic is coupled to the inert support through a non-peptidyl linker arm. U.S. Pat. No. 5,484,773 describes oligosaccharides sequences attached covalently attached to pharmaceutical solids, wherein said oligosaccharides sequences bind C. difficile toxin A, while U.S. Pat. No. 6,013,635 describes the same concept but targeted to C. difficile toxin B.

Another method of treating a C. difficile toxin mediated disorder comprises administeration to a subject in need thereof of an effective amount of a toxin binding composition comprising a toxin binding moiety and a polymeric particle. At least about 90% of C. difficile toxin A is bound by the composition at a concentration ranging from about 0.1 mg/mL to about 20 mg/mL. The C. difficile toxin A being treated with the toxin binding composition in a phosphate buffer solution containing about 5% fetal bovine serum. Preferably, the concentration of the toxin binding composition needed to bind about 90% of C. difficile toxin A is from about 0.5 mg/mL to about 10 mg/mL; more preferably, from about 0.8 mg/mL to about 5 mg/mL; even more preferably, from about 1 mg/mL to about 3 mg/mL. In another method of treating a C. difficile toxin mediated disorder comprises administration to a subject in need thereof of an effective amount of a toxin binding composition comprising a toxin binding moiety and a polymeric particle. At least about 90% of C. difficile toxin B is bound by the composition at a concentration ranging from about 0.1 mg/mL to about 20 mg/mL. The C. difficile toxin B being treated with the toxin binding composition in a phosphate buffer solution containing about 5% fetal bovine serum. Preferably, the concentration of the toxin binding composition needed to bind about 90% of C. difficile toxin B is from about 0.8 mg/mL to about 10 mg/mL; more preferably, from about 1 mg/mL to about 6 mg/mL.

In some of the various embodiments, the C. difficile toxin A and B are purified. Incubation of the C. difficile toxin A with toxin binding composition can be carried out for about 2 hours to about 36 hours; preferably, from about 4 hours to about 24 hours; more preferably from about 12 hours to about 18 hours. The incubation typically is carried out at a temperature ranging from about 30° C. to about 40° C.; preferably about 37° C. The amount of toxin bound to the polymeric particle was calculated from determining the amount of free toxin in the supernatant by C. difficile toxin ELISA and subtracting the amount of free toxin from the amount of C. difficile toxin added to the mixture. The values resulting from the tests are tabulated in Table 8 and described in more detail in Example 8.

Formulations, Routes of Administration, Dosage

The compositions described herein or pharmaceutically acceptable salts thereof, can be delivered to the patient using a wide variety of routes or modes of administration. The most preferred routes for administration are oral, intestinal, or rectal.

If necessary, the compositions may be administered in combination with other therapeutic agents. The choice of therapeutic agents that can be co-administered with the compounds of the invention will depend, in part, on the condition being treated.

The polymers (or pharmaceutically acceptable salts thereof) may be administered per se or in the form of a pharmaceutical composition wherein the active compound(s) is in admixture or mixture with one or more pharmaceutically acceptable carriers, excipients or diluents. Pharmaceutical compositions for use in accordance with the present invention may be formulated in conventional manner using one or more physiologically acceptable carriers compromising excipients and auxiliaries which facilitate processing of the active compounds into preparations which can be used pharmaceutically. Proper formulation is dependent upon the route of administration chosen.

When used for oral administration, which is preferred, these compositions may be formulated in a variety of ways. It will preferably be in freeze-dried, liquid, solid, or semisolid form. Compositions including a liquid pharmaceutically inert carrier such as water or castor oil may be considered for oral administration. Other pharmaceutically compatible liquids or semisolids, may also be used. The use of such liquids and semisolids is well known to those of skill in the art. See, e.g., Remington's Pharmaceutical Sciences, 18th edition, 1990.

Compositions which may be mixed with semisolid foods such as applesauce, ice cream or pudding may also be preferred. Formulations, which do not have a disagreeable taste or aftertaste, are preferred. A nasogastric tube may also be used to deliver the compositions directly into the stomach.

Solid compositions may also be used, and may optionally and conveniently be used in formulations containing a pharmaceutically inert carrier, including conventional solid carriers such as lactose, starch, dextrin or magnesium stearate, which are conveniently presented in tablet or capsule form. Capsules can also be liquid or gel containing capsules. The composition itself may also be used without the addition of inert pharmaceutical carriers, particularly for use in capsule form.

Typically, doses are selected to provide neutralization and elimination of the toxins found in the gut of the effected patient. Useful doses are from about 1 to 100 micromoles of oligosaccharide/kg body weight/day, preferably about 10 to 50 micromoles of oligosaccharide/kg body weight/day. The dose level and schedule of administration may vary depending on the particular oligosaccharide structure used and such factors as the age and condition of the subject.

As discussed previously, oral administration is preferred, but formulations may also be considered for other means of administration such as per rectum. The usefulness of these formulations may depend on the particular composition used and the particular subject receiving the treatment. These formulations may contain a liquid carrier that may be oily, aqueous, emulsified or contain certain solvents suitable to the mode of administration. Compositions may be formulated in unit dose form, or in multiple or subunit doses.

### EXAMPLES

### Example 1

### Synthesis of Toxin Binding Compositions

SM1 precursor 1 was synthesized as previously reported. See WO 02/044190. 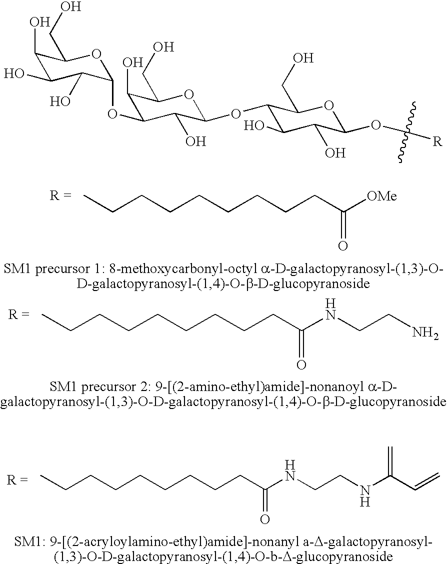
Synthesis of SM1:

To a solution of 25 mL ethylene diamine (370 mmol) and 30 mL of dimethylformamide, 10 gm of SM1 precursor 1 (14.8 mmol) was added and the reaction mixture was stirred at 85° C. for 18 hours. Progress of reaction was monitored by TLC (dichloromethane:methanol:water=6:4:0.15). Upon completion of reaction, the mixture was concentrated to 20 mL with rotary evaporator and the SM1 precursor 2 was obtained as white precipitate by pouring the concentrate into 1.5 L isopropanol. The filtered precipitate was dried under vacuum for 10 hours and used directly for subsequent acyloylation.

Crude SM1 precursor 2 was suspended in 80 mL MeOH/water mixture (1:1 by volume) and stirred in ice bath. 4.6 gm sodium carbonate (44 mmol) was added, which was followed by addition of 3.6 mL acryloyl chloride (44 mmol) with a dropping funnel over 10 minutes. The mixture was stirred from 0° C. to room temperature for 4 hours. Progress of reaction was monitored by TLC (dichloromethane:methanol:water=6:4:0.3). Upon completion of reaction, inorganic salts were filtering off and the filtrate was concentrated with rotary evaporator below 45° C. The acyloylated product SM1 (7.5 g, 10 mmol) was obtained by column chromatography purification (eluted with dichloromethane:methanol mixture from 5:1 to 2:1).

Synthesis of Block Copolymer:

To 0.25 gm SM1, 0.05 gm dimethylacrylamide and 7 mg dithioester RAFT agent were added 1.36 mL (1:1 by volume) water/dimethylformamide mixture, which was heated to 50° C. 0.98 mg of initiator, 2,2′-azobis[2-(2-imidazolin-2-yl)propane]dihydrochloride (VA-044 from Wako) in 30 μl water was added. Monomer conversion was tracked by proton NMR and molecular weight of polymer was obtained by GPC analysis. With >90% conversion of first block monomers after 2 to 3 hours, 0.27 gm n-butylacrylate was added semi-continuously over 3 hours. Upon completion of the butylacrylate addition, the reaction mixture was stirred for an additional 3 hours, then 8 mL water was added to the mixture, which was used directly for latex preparation or dialyzed in water prior emulsion polymerization.

Latex Preparation:

Ingredients:

| | | |
|---|---|---|
| SM1containing block | 10 | mL (0.57 gm polymer) |
| copolymer solution | | |
| Styrene | \tilde{1}2 | mL (0.\tilde{9}1.8 gm) |
| Potassium persulfate (KPS) | 2.\tilde{7}10 | mg |
| Deionized water | 3\tilde{4}68 | mL |

KPS stock:

50 mg of KPS in 2 mL deionized water

10 mL SM1 block copolymer solution, 30 mL water and 0.33 mL styrene were added to a 100 mL 3-neck morton flask. The reaction mixture was purged gently with argon and stirred with a magnetic bar at 700 rpm at room temperature for 2 hours. Polymerization was triggered by the addition of 108 μl KPS stock solution and increasing the temperature to 60° C. Remaining styrene (2×0.33 mL) was added at 1.5 and 3 hours after the KPS addition. After 6 hours polymerization, the temperature was brought to room temperature and the latex solution was filtered by glass wool and 0.45 μm GMF filter. Removal of residual monomers was accomplished by 10 day dialysis in deionized water.

This protocol was used to generate several type of particle suspensions with the same block copolymer but various monomer compositions. See Table 2.

TABLE 2

| Latex | | | | | | | | | |
|---|---|---|---|---|---|---|---|---|---|
| sample | tm387c | tm444a | tm444b | tm461a | tm461c | tm466d | tm473a | tm473b | tm475a |
| SM1 in 1st block (mg) | 250 | 250 | 250 | 250 | 250 | 250 | 250 | 250 | 250 |
| DMA in 1st block (mg) | 50 | 50 | 50 | 50 | 50 | 50 | 50 | 50 | 50 |
| BA in 2nd block (mg) | 270 | 270 | 270 | 270 | 270 | 270 | 270 | 270 | 270 |
| Wt of diblock (gm) | 0.57 | 0.57 | 0.57 | 0.57 | 1.33 | 0.57 | 0.57 | 0.57 | 0.57 |
| Wt of styrene (gm) | 1.8 | 1.8 | 1.8 | 1.8 | 1.8 | 1.8 | 1.8 | 0.9 | 1.8 |
| Wt of DVB (gm) | 0.146 | 0.219 | 0.219 | 0.146 | 0.146 | 0 | 0 | 0 | 0 |
| Total wt (gm) | 2.516 | 2.589 | 2.589 | 2.516 | 3.276 | 2.37 | 2.37 | 1.47 | 2.37 |
| Final vol (ml) | 40 | 40 | 70 | 45 | 45 | 40 | 40 | 40 | 40 |
| Latex radius (nm) | 78 | 114 | 165 | 125 | 245 | 88 | 89 | 67 | 86 |
| Solid content % | 4.4 | 3.3 | 1.7 | 3.7 | 1.7 | 3.3 | 4.3 | 3 | 4.7 |
| Total mass of latex (gm) | 1.76 | 1.32 | 1.19 | 1.665 | 0.765 | 1.32 | 1.72 | 1.2 | 1.88 |
| Total vol of latex (ml) | 1.848 | 1.386 | 1.2495 | 1.74825 | 0.80325 | 1.386 | 1.806 | 1.26 | 1.974 |
| Vol of single latex particle | 1.99E−15 | 6.21E−15 | 1.88E−14 | 8.18E−15 | 6.16E−14 | 2.85E−15 | 2.95E−15 | 1.26E−15 | 2.66E−15 |
| (cm³) | | | | | | | | | |
| Total no of latex particles (N) | 9.30E+14 | 2.23E+14 | 6.64E+13 | 2.14E+14 | 1.30E+13 | 4.86E+14 | 6.12E+14 | 1.00E+15 | 7.41E+14 |
| Surface area of latex (m²) | 7.65E−14 | 1.63E−13 | 3.42E−13 | 1.96E−13 | 7.54E−13 | 9.73E−14 | 9.95E−14 | 5.64E−14 | 9.29E−14 |
| Total latex area of sample | 71 | 36 | 23 | 42 | 10 | 47 | 61 | 56 | 69 |
| (m²) | | | | | | | | | |
| SM1 micromoles | 231.0 | 168.4 | 151.8 | 218.5 | 77.1 | 183.9 | 239.7 | 269.6 | 262.0 |
| SM1 surface density | 3.3 | 4.6 | 6.7 | 5.2 | 7.8 | 3.9 | 3.9 | 4.8 | 3.8 |
| (micromoles/m²) | | | | | | | | | |
| SM1 micromoles per gm latex | 131.3 | 127.6 | 127.6 | 131.3 | 100.8 | 139.3 | 139.3 | 224.7 | 139.3 |

Total mass of latex = solid content w/v % × volume of latex solution
Total volume of latex = 1.05 × total mass of latex (Based on assumption for the density of styrene latex 1.05 gm/ml)
Latex particle size = 4/3 × pi × (power 3 of measured latex radius)
Total no of latex = total volume of latex/volume of a latex particle
Surface area of a latex particle = 4 × pi × (power 2 of latex radius)
Total latex surface area = total no of latex × Surface area of a latex particle
Total SM1 fed (micromoles) = 250 * 10\hat{ }6/(1000 * 757) (the molecular weight of SM1 = 757) When calculating micromoles/square meter and per gram, this figure is adjusted for actual yield
RAFT = Reverse-addition fragment transfer reagent used for controlling the size of growing polymer and retaining the propagating property of the polymer
KPS = Potassium persulfate as an aqueous soluble initiator to trigger the polymerization process
DVB = divinylbenzene as a crosslinking formulation ingradient to enhance the stability of latex
Solid content = express as % (g/dL) related to the latex concentration delivered to biology assay

The surface density of saccharide present at the particle surface was computed as follows:
Surface density (microequivalents/m²)=microequivalents of sugar/gm of solid *(6/(d*D))⁻¹, where d is the density of the polymer particle and D is the particle diameter in microns.
Latex Preparation—Nonionic Initiator

General Recipe:

| | | |
|---|---|---|
| 1. SM1containing block | 7 | mL (0.39 gm diblock |
| copolymer solution | | polymer) |
| 2. Styrene | \tilde{1}1.2 | mL |
| 3. Hydrogen peroxide | 5.8 | mg |
| 4. Ascorbic acid | 5.6 | mg |
| 5. Deionized water | 35 | mL |

Recipe of H₂O₂ stock solution: 33 μl of 30 wt % hydrogen peroxide was added to 167 μl deionized water

Recipe of ascorbic acid stock solution: 10 mg ascorbic acid was added to 2 mL deionized water.

Reaction procedure: 7 mL of SM1 block copolymer solution, 35 mL water and 0.3 mL styrene were stirred at 700 rpm at room in a 100 mL three-neck Morton flask under nitrogen for 2 hours. Subsequently, the reaction mixture was heated to 60° C. over 2 hours. Then 116 mL H₂O₂ stock solution and 112 mL ascorbic acid stock solution were added to the mixture. After stirring at 60° C. for 60 minutes, the remaining styrene (0.7 to 0.9 mL) was added semi-continuously over 240 minutes, every 40 minutes. The reaction mixture was stirred for 2 more hours upon the completion of styrene addition cycle, then the temperature was brought to room temperature and the latex solution was filtered by 25 μm pore size filter paper. Removal of residual monomers was accomplished by 10 day dialysis in deionized water.

Synthesis of SM1 Containing Mesoporous Hydrogel

| | |
|---|---|
| SM1 monomer | 0.228 gm |
| Vinylformamide | 0.027 gm |
| Benzylacrylamide | 0.046 gm |
| N,N′-ethylene bisacrylamide | 25 or 50 mg |
| Types of porogens | water/DMF/n-butanol |
| | (3:3:4 or 2:2:3 volme ratio) or |
| | water/DMF/n-hexanol |
| | (3:3:4 or 2:2:3 volme ratio) |
| Volume of porogen | 1 to 1.5 mL |
| VA-044 (2,2′-azobis[2,(2- | 1.5 mg |
| imidazolin-2-yl) | |
| Propane] hydrogen chloride | |
| Stirrer type and speed | 12 mm magnetic flea/1000 rpm |
| Reaction vessel | Kimble auto sampler 4 mL vial |

Preparation of hydrogel was performed in Glove box with oxygen level below 10 ppm. To 0.325 or 0.35 gm of monomers (SM1, vinylformamide, benzylacrylamide and N,N′-ethylene bisacrylamide), 1.3 mL of porogen and 1.5 mg of VA-044 was added. The mixture was stirred overnight at 50° C. and a white opaque rubber-like solid was obtained, which was milled into micro-particle suspension in 8 mL water by 3 minutes sonication. The suspension was dialyzed in DI water for 2 days and dried over lyophilizer (2 days).

### Example 2

### In-vitro (ELISA and Cell Culture) Assays

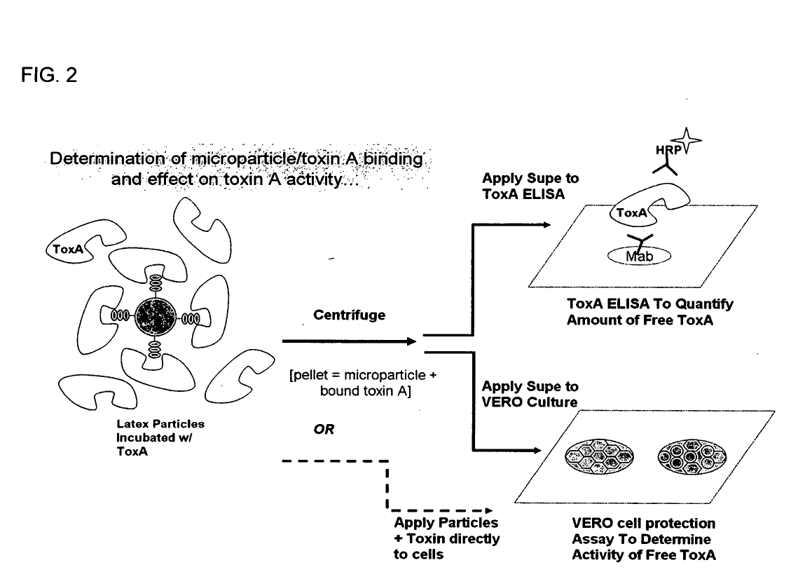

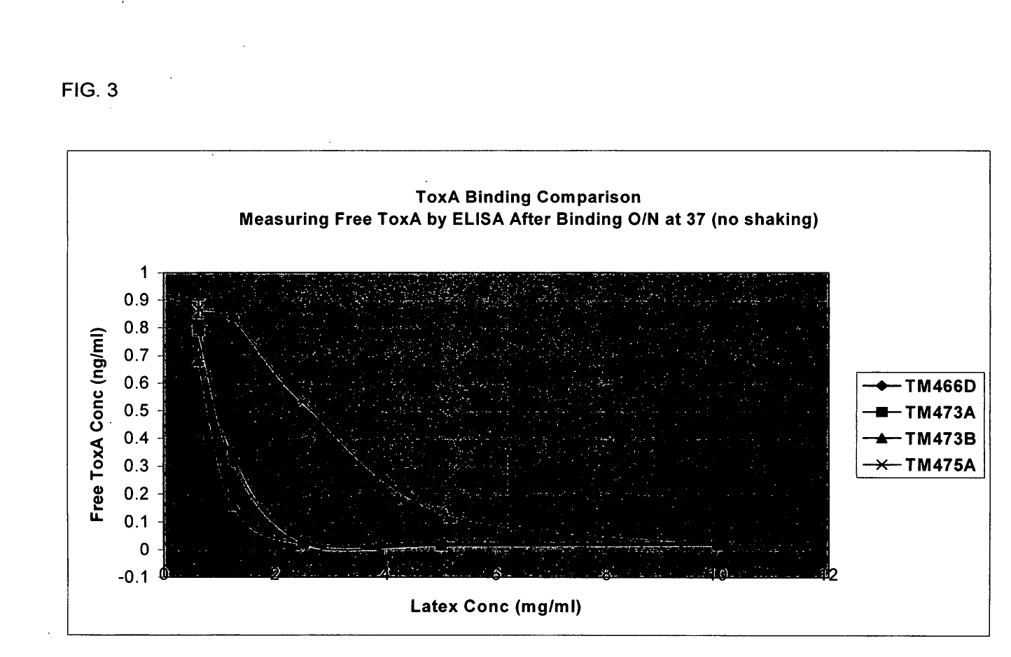

Two in vitro assays were used to measure the toxin binding and neutralization properties of the microparticles synthesized in Example 1. FIG. 2 depicts a summary of ELISA and tissue culture assays used to measure bioactivity of toxin molecules treated with micro-particles. In the toxin ELISA assay, the micro-particles (test concentrations ranging from 1-10 mg/mL) are incubated with toxin (concentration of 1 ng/mL to 160 μg/mL) at 37° C. with no shaking of the mixture. After an 18-hour incubation, the micro-particle/toxin mixture is centrifuged to remove pelleted material representing complexes of the micro-particles and bound toxin. The supernatant from this centrifugation step contains unbound toxin molecules, which are quantified by a standard ELISA assay consisting of PCG-4 monoclonal antibody to “capture” the unbound toxin molecules and a horse radish peroxidase-conjugated polyclonal antibody that is used to detect the immobilized toxin molecules. See Lyerly, D. M., C. J. Phelps, J. Toth, and T. D. Wilkins. 1986. Characterization of toxins A and B of Clostridium difficile with monoclonal antibodies. Infect Immun. 54:70-6. A representative ELISA profile for four distinct micro-particle compositions is presented in FIG. 3. The materials TM473B, TM473A, and TM466D reduced free toxin A (1 ng/mL starting concentration) in the incubation mixture by >50% at the lowest concentration of microparticle tested (1 mg/mL). The IC90s of different microparticles (the concentration of microparticle where 90% of the toxin is removed from the supernatant) with starting concentrations of C. difficile Toxin A and Toxin B are shown in Table 2.

Cell culture assays with mammalian epithelial cells represent a second method used to evaluate bioactivity of the unbound toxin molecules after incubation with test micro-particles. In this assay, the VERO cell line (African Green Monkey kidney epithelial cells) was cultured in standard 96-well tissue culture format and overlayed with dilutions of the supernatant obtained from the centrifugation step following micro-particle/toxin mixture (as described above). Combined with the ELISA measurement to quantify free, unbound toxin, this assay provides a measure of bioactivity for the unbound toxin. In all cases, pretreatment with the micro-particles did not inactivate the remaining unbound toxin, as measured by the cell culture assay.

The cell culture assay is also used to quantify the degree of neutralization provided by the micro-particles when mixed with toxin. In this assay, various concentrations of the micro-particles (1-20 mg/mL) are mixed with a fixed amount of toxin (0.3 pg/mL-1 ng/mL) that is known to cause “cell rounding” (i.e., a cytotoxic effect that disrupts normal adherence of the cells to the plastic surface, usually indicating cell death or loss of intracellular filament structure). In some cases, the microparticles were kept from coming into direct contact with the cells by using transwells with a semi-permeable membrane (i.e. permeable to Toxin). This was to show that microparticle-cell contact was not required for protection of the cells from Toxin effect. The relative extent of toxin neutralization is compared by microscopic examination of multiple cell fields (>10), quantifying the % of rounded cells in the background of confluent cell growth. The lowest effective micro-particle dose that results in >95% protection from cell rounding is used to provide a measure of micro-particle activity. The data is provided in Table 3.

TABLE 3
Summary of representative VERO cell screening and ELISA data using various
compositions of SM1-containing micro-particles; reported as lowest effective micro-
particle dose resulting in >95% protection from cell rounding or >90% removal of
Toxin from solution.

| | | | | [microparticle] | | | |
|---|---|---|---|---|---|---|---|
| | | | | resulting in 95% cell | | [microparticle] | |
| | | | | protection from | | resulting in 90% | |
| | | | | Toxin A | | removal of Txin from | |
| | | | | With | Without | s/nat (ELISA) | |
| | | | Solid | Transwells | Transwells | Toxin A | Toxin B |
| | Radius | | Content | (2 ng/ml | (1 ng/ml | (10 μg/ml | (10 μg/ml |
| Microparticle | (nm) | Initiator | (%) | Toxin) | Toxin) | [starting]) | [starting]) |
| tm387c | 78 | Potassium | 6 | 4 | 5 | nm | nm |
| | | Persulfate | | | | | |
| tm444a | 114 | Potassium | 3.3 | 3.3 | nm | nm | nm |
| | | Persulfate | | | | | |
| tm444b | 165 | Potassium | 1.7 | 2.2 | nm | nm | nm |
| | | Persulfate | | | | | |
| tm461a | 125 | Potassium | 3.7 | 4.6 | 10 | nm | nm |
| | | Persulfate | | | | | |
| tm461c | 245 | Potassium | 1.7 | 1.7 | nm | nm | nm |
| | | Persulfate | | | | | |
| tm466d | 88 | Potassium | 3.3 | 2.2 | 4.1 | 2.3 | >5 |
| | | Persulfate | | | | | |
| tm473a | 89 | Potassium | 4.3 | 2.9 | 5.4 | 1.8 | >5 |
| | | Persulfate | | | | | |
| tm473b | 67 | Potassium | 3 | 2 | 3.8 | 1.9 | 4.5 |
| | | Persulfate | | | | | |
| tm475a | 86 | Potassium | 4.7 | 2.3 | nm | nm | nm |
| | | Persulfate | | | | | |
| tilm149a | 111 | H₂O₂/ | 1.6 | nm | nm | <<1.9 | <1.94 |
| | | Ascorbate | | | | | |

nm: not measured

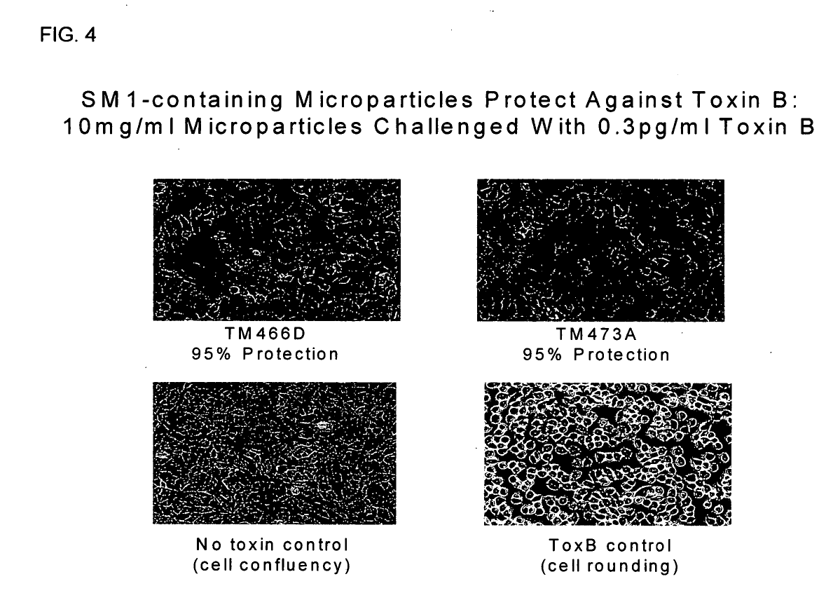

The SM1-containing micro-particles were also able to neutralize toxin B activity. Using the method described above, the micro-particles provided >95% protection against a 0.3 pg/mL challenge dose of toxin B when used at a 10 mg/mL dose (see FIG. 4).

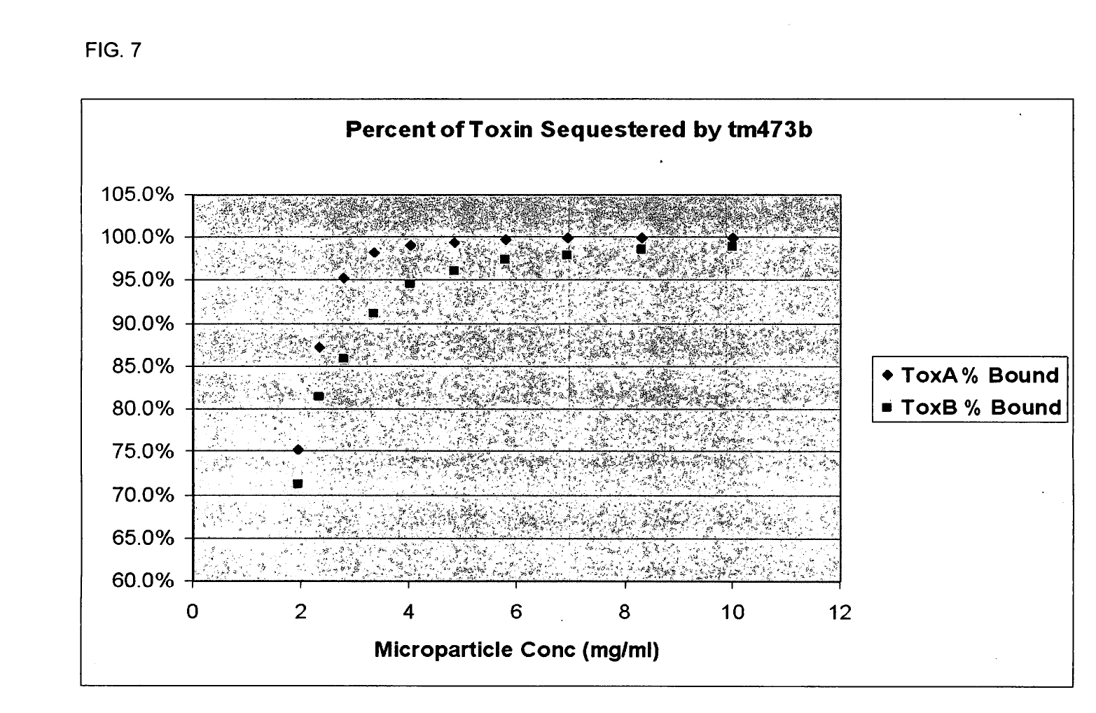

FIG. 7 shows the percent of toxin A and B bound by a range of concentrations for the microparticle, tm473b.

### Example 3

### Binding Capacity of Microparticles (TM473B)

One of the microparticle samples of Example 1, TM473B, was made into 2× solutions at 20, 10, 5, and 2.5 mg/mL concentrations by diluting the microparticles in blocking buffer (1× Phosphate-buffered saline with 5% Fetal Bovine Serum). Purified C. diff Toxin A and B (TechLab T3001 and T3002) were diluted in blocking buffer to 2× solutions ranging from 360-2 μg/mL. In a checkerboard fashion, the dilutions were mixed into a final 1:1 ratio of microparticles to toxin.

To allow the microparticles to reach equilibrium binding, the samples were incubated at 37° C. for 18 hours. Bound Toxin A or B was pelletted with the microparticles by centrifuging at 10,000 rpm for 1 hour. Supernatant containing free/equilibrium toxin was collected and the concentration was determined by Toxin A or Toxin A and B ELISA Kits (TechLab C. Diff Tox-A Test T5001 or C. Diff Tox-A/BII Test T5015).

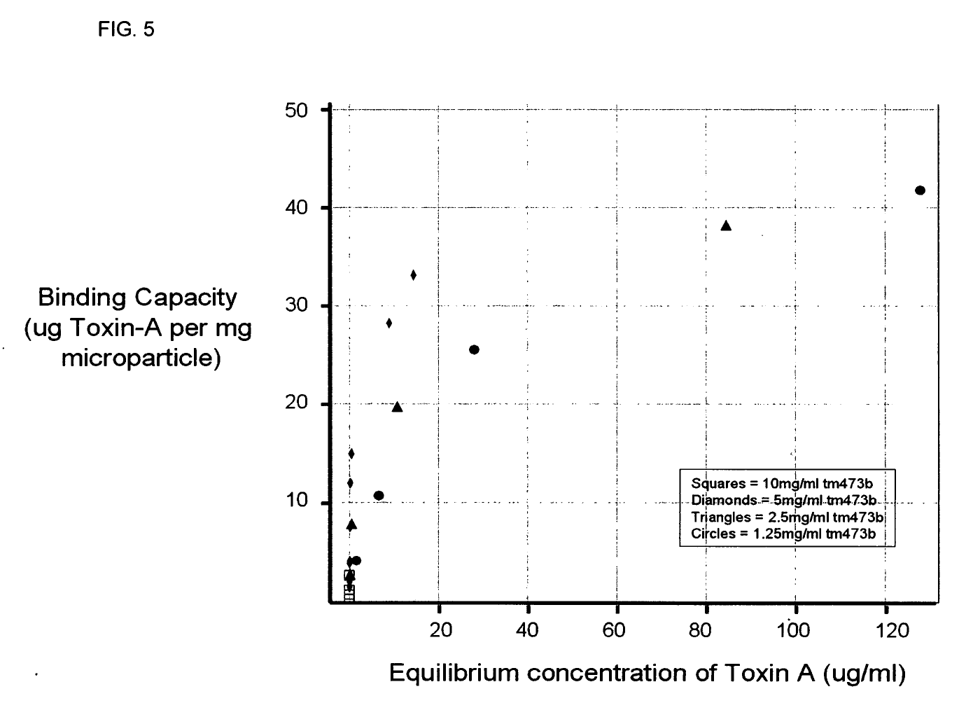

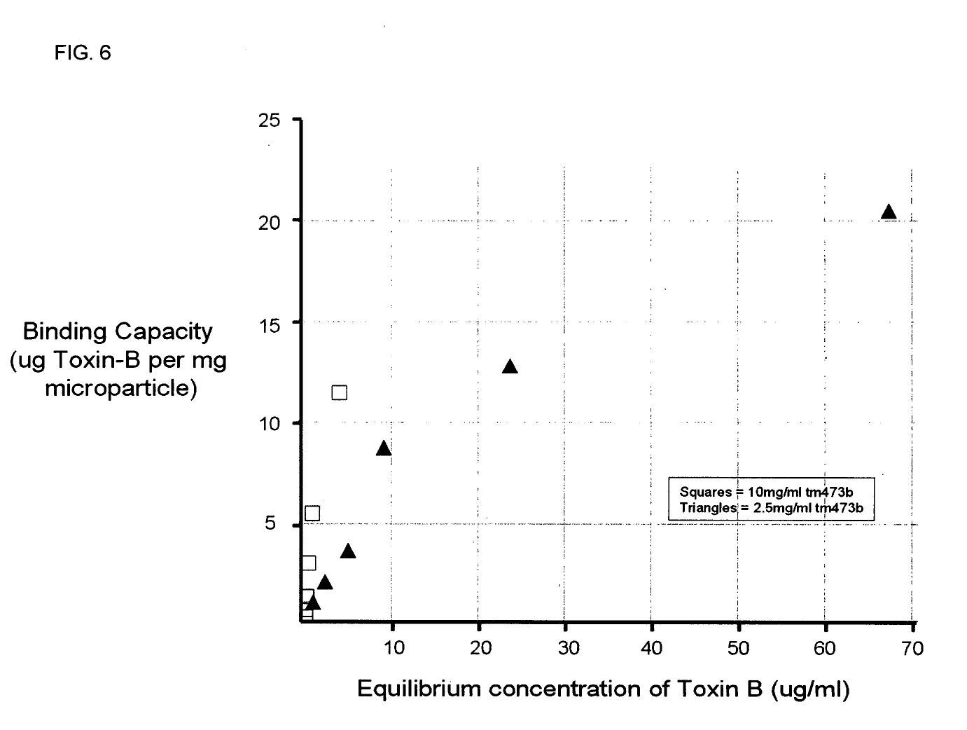

To determine the concentration of bound toxin, the equilibrium concentration was subtracted from the starting amount. Binding capacities were then calculated by dividing the concentration of bound toxin in μg/mL by the microparticle concentration in mg/mL. The results are provided in FIGS. 5 and 6.

### Example 4

### In-vivo Testing of Microparticle Efficacy: Rabbit Ileal Loop Toxicity Test

Two of the microparticle samples of Example 1, TM473A and TM473B, were tested in vitro in a rabbit ileal loop model study. The rabbit ileal loop model is a model for demonstrating enterotoxicity of bacterial protein toxins (Duncan and Strong, 1969). The model has been used to characterize enterotoxic activity of cholera toxin, E. coli labile toxin, shiga toxin, and various clostridial toxins including C. perfringens enterotoxin and C. difficile toxin A.

The protocol for the rabbit ileal loop test of C. difficile toxin A is as follows:
Rabbits (of either sex, >12 weeks of age) were fasted overnight and then anaesthetized with 0.25 mL of ketamine hydrochloride (100 mg/mL) mixed with 0.25 mL of diazepam (5 mg/mL) injected intravenously in the marginal ear vein.
Anesthesia was maintained using halothane (1.5-2.5% to effect), nitrous oxide (21/min flow) and oxygen (11/min) delivered via a gas anesthesia machine.
The mid-section of each anaesthetized rabbit's abdomen was shaved and prepared aseptically using a series of alternating betadyne and isopropyl alcohol scrubs, and a 5 cm abdominal incision was made.
The ileum was carefully withdrawn, and up to 6 ileal loops (˜7-10 cm long), ˜1 cm apart were constructed by sealing a section of ileum at each end with a sterile cotton ligature.
Fluid (0.5 mL/loop) containing a mixture of test micro-particle (upto 20 mg/mL) and toxin A (10 μg/mL) was injected through a 26-gauge needle into each test loop at a location about 0.5 cm immediately below the single proximal ligature.
The injection site was isolated to prevent leakage by a further ligature about 0.5 cm distally of the puncture site.
After inoculation of the loops, the ileum was again moistened with warm saline and gently returned to the abdominal cavity. After suturing the muscle wall and closing the skin incision, the animals were kept warm and monitored during the anesthetic recovery period. Oxymorphone (0.25 mL i.m./rabbit; 1.5 mg/mL) was given before anesthetic recovery and again at 6-8 h after surgery. Food and water was withheld post-operatively.
Approximately 8-12 hour after surgery, the rabbits were euthanized with a 0.5-1.0 mL intravenous injection of Beuthanasia D (390 mg/mL pentobarbital, 50 mg/mL phenytoin).
The ileum was removed, and fluid accumulation in individual loops was assessed visually. The length and weight of each positive loop was then measured and its contents weighed for calculating the V/L (volume-length) ratio, which is the ratio of weight of loop contents in grams to loop length in centimeters.
Positive loops (those accumulating fluid) were defined as having V/L ratios >0.3 and containing a serosanguinous fluid with a free-flowing, watery consistency. Negative loops had no recoverable content, i.e. those loops with V/L ratios <0.1.

Using this protocol, microparticles test samples TM473B and TM473A provided protection against toxin A (10 microgm/mL) enterotoxicity when dosed at 2.5 mg/mL. See Table 4.

TABLE 4
Rabbit Ileal Loop Tests

| | Concentration of | Toxin A | # of | |
|---|---|---|---|---|
| | microparticle | (microgm/ml) | loops | # of loops |
| Microparticle | tested (mg/ml) | challenge | tested | protected |
| TM473A | 20 | 10 | 8 | 8 |
| | 10 | 10 | 3 | 3 |
| | 5 | 10 | 3 | 3 |
| | 2.5 | 10 | 3 | 2 |
| | 1 | 10 | 2 | 0 |
| TM473B | 20 | 10 | 16 | 14 |
| | 10 | 10 | 3 | 3 |
| | 5 | 10 | 3 | 3 |
| | 2.5 | 10 | 3 | 2 |
| | 1 | 10 | 1 | 0 |
| | 0.5 | 10 | 1 | 0 |

### Example 5

### Preparation and Testing of Diblock Micelles and Microparticles Having Lower Carbohydrate Monomer Content

In a further set of experiments, additional formulations of diblock copolymer micelles and corresponding microparticles—having lower carbohydrate monomer (SM1) content—were prepared and evaluated in vitro. Specifically, diblock copolymers were prepared comprising about 33% by weight carbohydrate monomer SM1 (referred to herein as “diblock copolymer A”), and separately, comprising about 21% by weight carbohydrate monomer SM1 (referred to herein as “diblock copolymer B”). The diblock copolymers had substantially lower carbohydrate monomer content than the diblock copolymer prepared as described in Example 1, in which carbohydrate monomer SM1 constituted about 44% by weight. A micelle solution formed from the diblock copolymer B was subsequently evaluated in vitro in a cell culture assay. Also, latex microparticles were synthesized from each of the diblock copolymer A and the diblock copolymer B, and were also evaluated in vitro.

Preparation of Diblock Copolymers A and B

Carbohydrate monomer, SM1, was prepared substantially as described in Example 1. Two different formulations of diblock copolymers—having relatively lower carbohydrate monomer (SM1) content—were prepared as follows, using reagents and amounts as described in Table 5.

TABLE 5
Formulations for Diblock Copolymers A and B

| | | | Wt % of | | | Wt % of |
|---|---|---|---|---|---|---|
| | Amount of | | monomer | Amount of | | monomer |
| | reagent in | | in diblock | reagent in | | in diblock |
| Reagents | copolymer A | | copolymer A | copolymer B | | copolymer B |
| THMA (93%) | 90 | mg | 7 | 183 | mg | 15 |
| Carbohydrate | 0.37 | g | 33 | 0.24 | g | 21 |
| monomer | | | | | | |
| (SM1) | | | | | | |
| DMA | 0.14 | g | 12 | 0.19 | g | 17 |
| n-butylacrylate | 0.6 | mL | 48 | 0.6 | mL | 47 |
| CTA | 14 | mg | | 14 | mg | |
| VA-044 | 1 | mg | | 1 | mg | |
| Water/DMF | 3.4 | mL | | 3.4 | mL | |

THMA = N-[Tris(hydroxymethyl)-methyl]acrylamide, 93% purity
CTA = 2-(3,5-Dimethyl-pyrazole-1-carbothioylsulfanyl)-propionic acid ethyl ester
VA-044 = 2,2′-azobis[2-(2-imidazolin-2-yl)propane]dihydrochloride
DMA = dimethylacrylamide

For each diblock copolymer A and B, to a mixture of THMA, DMA, carbohydrate monomer (SM1) and CTA was added 3.4 mL water/DMF mixture and heated to 50° C. in a glove box. 20 μL of VA-044 stock solution (20 mg in 0.2 mL DI water) was added and the mixture was stirred for 3 hours. n-Butylacrylate was added semi-continuously over the next 3 hours and followed by additional 3 hour polymerization before cooling the mixture to room temperature. 10 mL of DI water was added to mixture and followed by dialysis of the diblock copolymer in DI water for 24 hours, to form diblock micelle solutions.

In-Vitro Cell Culture Assay of Diblock Copolymer B Micelles

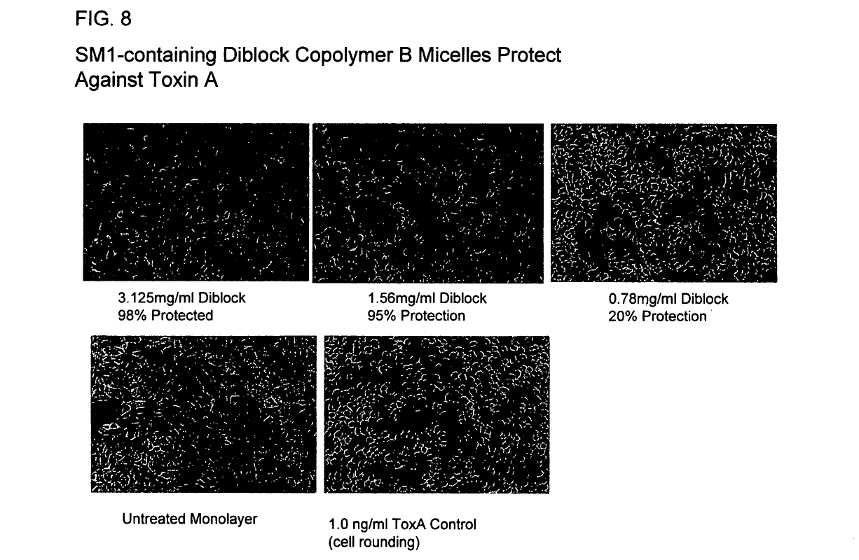

A micelle solution formed from the diblock copolymer B was evaluated in vitro in a cell culture assay. In this assay, VERO cells were treated with solutions having various concentrations of the diblock copolymer B micelles (0.78 mg/mL, 1.56 mg/mL and 3.125 mg/mL), in each case mixed with a fixed amount of C. diff. toxin A (1 ng/mL). The fixed amount of toxin was known to cause “cell rounding” (i.e., a cytotoxic effect that disrupts normal adherence of the cells to the plastic surface, usually indicating cell death or loss of intracellular filament structure) when used by itself. As one control, a monolayer of untreated VERO cells (i.e., untreated with toxin or with diblock copolymer B micelles) was used. As another control, VERO cells treated only with C. diff. toxin A (1 ng/mL) was used. The relative extent of toxin neutralization was compared by microscopic examination of multiple cell fields (>10), quantifying the % of rounded cells in the background of confluent cell growth. The results are shown in FIG. 8.

Preparation of Microparticles Using Diblock Copolymers A and B

Latex microparticles were synthesized from each of the diblock copolymer A and the diblock copolymer B, using reagents and amounts as described in Table 6. The resulting microparticles are referred to herein as glycoparticles A and B, respectively, and are also designated as tilm209A and tilm209B, respectively.

TABLE 6
Formulations for Microparticles A and B

| | Glycoparticles A | | glycoparticles B | |
|---|---|---|---|---|
| Reagents | (tilm209A) | | (tilm209B) | |
| Source of micelle solution | diblock copolymer A | | diblock | |
| | | | copolymer B | |
| Volume of micelle solution | 14 mL at 55 mg/mL | | 14 mL at 53 mg/mL | |
| | polymer conc. | | polymer conc. | |
| Styrene | 2.4 | mL | 2.4 | mL |
| Potassium persulfate | 6 | mg | 6 | mg |
| (KPS) | | | | |
| DI water | 70 | mL | 70 | mL |

From each of the diblock copolymer A and B solutions, microparticles A (tilm209A) and B (tilm209B) were separately synthesized as follows. To a 250 mL 3-neck Morton flask, a diblock solution, 0.8 mL styrene and 70 mL DI water were added and stirred at room temperature under nitrogen overnight. The temperature was increased to 60° C. and stirred for additional 4 hours before the addition of 240 μL KPS stock solution (25 mg in 1 mL DI water). 1.6 mL styrene was added over the following 5 hours. 10 μL t-butyl peroxy benzoate and 25 mg of ascorbic acid were added 2 hours after the completion of styrene addition. The polymer mixture was continuously stirred for next 10 hours and filtered through a 25 μm pore size filter paper (from Whatman). The resulting glycoparticle suspension was dialyzed in DI water for 10 days.

In-Vitro ELISA Assay of Microparticles A and B

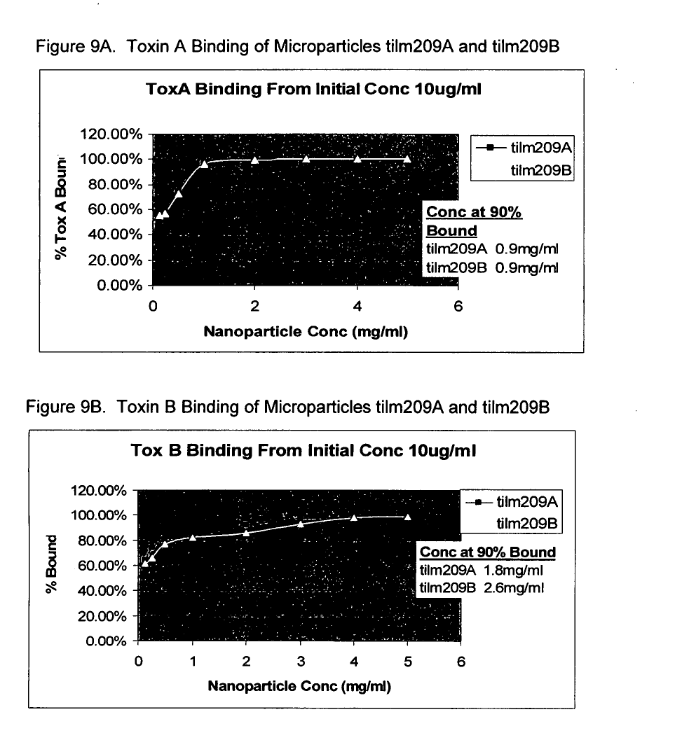

In-Vitro ELISA assays were used to determine the percentage of Toxin A and B bound by microparticles A (tilm209A) and B (tilm209B). In separate experiments for each of microparticles A and B, microparticles at concentrations ranging from 5 to 0.25 mg/mL in PBS containing 5% FBS were incubated with purified C. difficile toxin A or B (TechLab) at 10 μg/mL for 12-18 hours at 37° C. The mixtures were centrifuged at 10,500 rpm at 4° C. for 30 minutes, precipitating complexes of bound toxin with microparticles. The amount of free toxin remaining in the supernatants was determined by a toxin A-specific ELISA (TechLab #T5001 C. diff Tox-A Test) or a toxin A and B-specific ELISA (TechLab #T5015 C. diff Tox-A/BII Test). The results are shown in FIGS. 9A and 9B.

In-Vitro Cell Culture Assay of Microparticle B

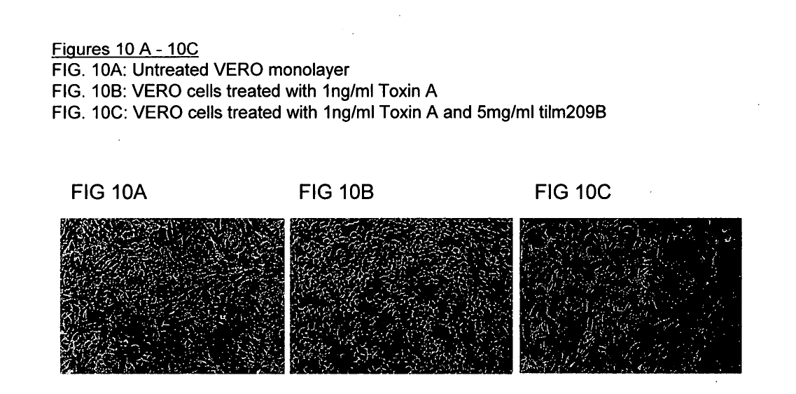

Microparticle B (tilm209B) was evaluated in vitro in a cell culture cytotoxicity assay. In this assay, confluent monolayers of VERO cells (ATCC) were grown in 96-well plates with MEM (Mediatech) supplemented with 10% fetal bovine serum. Purified C. difficile toxin A (TechLab) at a final concentration of 1 ng/mL was mixed with tilm209B microparticles at 5 mg/mL in growth medium and applied to the monolayers for 18 hours at 37° C., 5% CO2/95% air. Following incubation, the cells were examined microscopically for toxin-mediated morphological changes, identified by disruption of the monolayer and cell rounding. The results, shown in FIGS. 10A through 10C, demonstrate that the effects of 1 ng/mL Toxin A on VERO cells is neutralized by tilm209B at 5 mg/mL.

### Example 6

### In-Vitro Competitive Binding Experiment—Specificity of Galα(1,3)Galβ(1,4)Glc

In this example, a set of experiments involving in-vitro competitive binding assays were performed to demonstrate the specificity of C. diff. toxin-binding microparticles prepared substantially as set forth in Example 1.

In separate experiments, four different free oligosaccharides—including the trisaccharide Galα(1,3)Galβ(1,4)Glc, its isomer globotriose (Galα(1,4)Galβ(1,4)Glc), lactose (Galβ(1,4)Glc), and cellobiose (Glcβ(1,4)Glc)—were each assayed for their ability to compete with the C. diff. toxin-binding microparticles for toxin A and toxin B binding. The free oligosaccharides were tested at concentrations ranging from 6.25 mM to 50 mM, in each case against 2 mg/mL toxin-binding microparticles for binding to 10 μg/mL toxin A or toxin B. Mixtures were incubated for 16 hours at 37° C. Microparticles with bound toxin were precipitated by centrifugation and the amount of free toxin in the supernatant was determined by ELISA (TechLab).

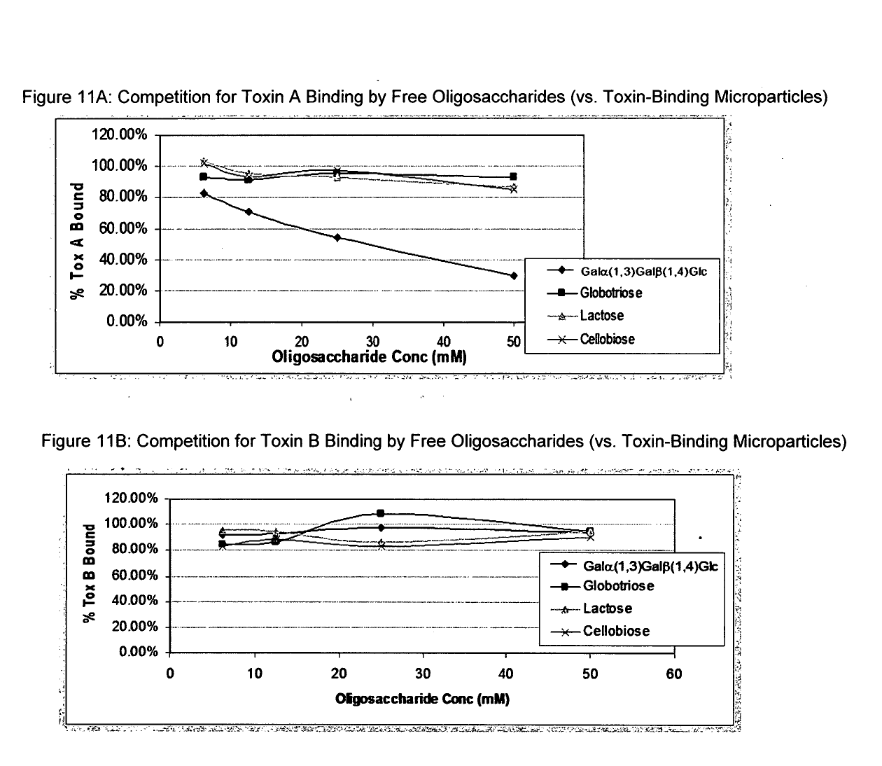

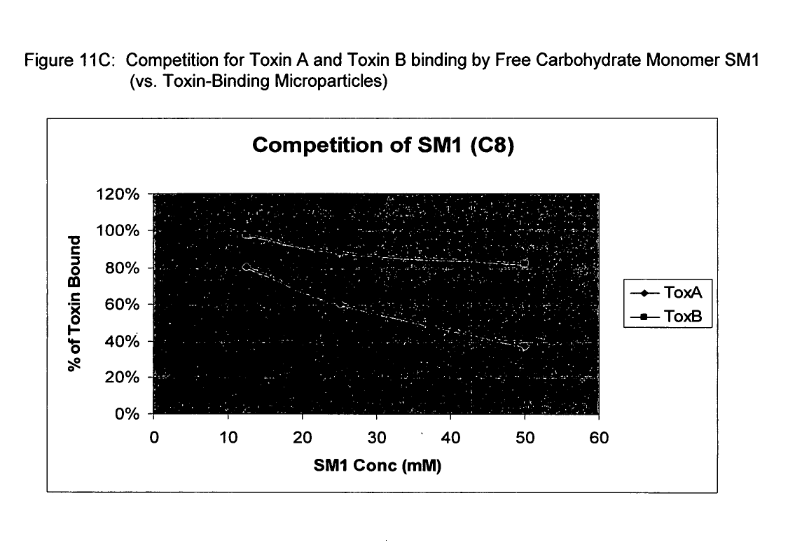

The results from these competitive binding experiments are shown in FIGS. 11A and 11B. Referring to FIG. 11A, the C. diff. toxin A preferentially binds to the toxin-binding microparticles over the free oligosaccharides globotriose (Galα(1,4)Galβ(1,4)Glc), lactose (Galβ(1,4)Glc), and cellobiose (Glcβ(1,4)Glc)—even at relatively high concentrations of such oligosaccharides. In contrast, the extent of binding of C. diff. toxin A by the toxin-binding microparticles varied depending on the concentration of the free trisaccharide Galα(1,3)Galβ(1,4)Glc. These data demonstrate the specific nature of the toxin-binding microparticle:Toxin A interaction, and confirm that toxin A binding by the microparticle specifically mediated by Galα(1,3)Galβ(1,4)Glc ligands. In contrast, FIG. 11B shows that toxin B preferentially binds to the toxin-binding microparticles over each of the four free oligosaccharides tested: the trisaccharide Galα(1,3)Gaβ(1,4)Glc, globotriose, lactose and cellobiose—even at relatively high concentrations of such oligosaccharides. Hence, although toxin B is bound by the toxin-binding microparticles (see FIG. 7, and related discussion in Example 2), the toxin B binding does not appear to be mediated directly through the Galα(1,3)Galβ(1,4)Glc ligands of the toxin-binding microparticles, since the free trisaccharide Galα(1,3)Galβ(1,4)Glc (nor any of the other three oligosaccharides) competed successfully with the microparticles for binding the toxin B.

In further experiments, the carbohydrate monomer SM1 (αGal-C8-linker; prepared substantially as set forth in Example 1) was likewise assayed for its ability to compete with the C. diff. toxin-binding microparticles for toxin A binding and for toxin B binding. The SM1 monomer was tested at concentrations ranging from 12.5 mM to 50 mM against 2 mg/mL toxin-binding microparticles binding to 10 μg/mL toxin A or toxin B. Mixtures were incubated for 16 hours at 37° C. Microparticles with bound toxin were precipitated by centrifugation and the amount of free toxin in the supernatant was determined by ELISA (TechLab).

The results are shown in FIG. 11C. As expected based on the results discussed above in connection with FIGS. 11A, toxin A binding by the microparticle is competitively mediated by Galα(1,3)Galβ(1,4)Glc ligands—present on both the microparticle and on the carbohydrate monomer SM1. With respect to toxin B binding, FIG. 11C shows that at higher concentrations, the carbohydrate monomer SM1 can compete with the toxin-binding microparticle to bind toxin B. Without being bound by theory, this provides some evidence that the interaction between the toxin-binding glycoparticles and C. diff. toxin B is mediated at least partially by a hydrophobic moiety (e.g., of the carbohydrate monomer SM1) (since no mediation was seen in the data of FIG. 11B involving free oligosaccharides), or by a combination of the trisaccharide ligand and a hydrophobic moiety (e.g., of the carbohydrate monomer SM1).

### Example 7

### In-Vivo Hamster C. difficile Challenge Study

In this example, an in-vivo hamster model was used to test toxin-binding microparticles prepared substantially as set forth in Example 1 (designated herein as Y103A2) for treatment of C. difficile-associated diarrhea.

Hamster Model

Generally, it is known that administration of antibiotics to hamsters prior to exposure to C. difficile results in diarrhea, colitis and eventually death after three to five days. Enterocolitis caused by C. difficile in hamsters occurs in the caecum and terminal ileum, characterized by mucosal epithelial cell proliferation and degenerative surface changes on the cells, along with mucosal hemorrhage; in contrast the human disease presents in the colon as focal crypt necrosis, with exudation and inflammation (Price et al., 1979) (full cite below). Despite these histological differences, the bacterial origin of C. difficile-associated diarrhea and its dependence on toxin A and B secretion for active disease makes the hamster model a suitable mimic of the human disease. (Bartlett et al., 1978a; Bartlett et al., 1978b; Chang et al., 1978). See:
Bartlett, J., C. T W, and G. M. 1978a. Antibiotic-associated pseudomembranous colitis due to toxin-producing clostridia. New England Journal of Medicine. 298:531-534.
Bartlett, J. G., T. W. Chang, N. Moon, and A. B. Onderdonk. 1978b. Antibiotic-induced lethal enterocolitis in hamsters: studies with eleven agents and evidence to support the pathogenic role of toxin-producing Clostridia. Am J Vet Res. 39:1525-30.
Chang, T. W., J. G. Bartlett, S. L. Gorbach, and A. B. Onderdonk. 1978. Clindamycin-induced enterocolitis in hamsters as a model of pseudomembranous colitis in patients. Infect Immun. 20:526-9.
Price, A. B., H. E. Larson, and J. Crow. 1979. Morphology of experimental antibiotic-associated enterocolitis in the hamster: a model for human pseudomembranous colitis and antibiotic-associated diarrhoea. Gut. 20:467-75.
   Hamster Strain and Numbers

In these experiments, hamsters were obtained from Harlan Laboratories, and held in quarantine for 7 days before treatment began. After quarantine, hamsters were weighed and randomly assigned to four groups. As summarized in Table 7, below, Group 1 was a control group that contained 6 animals. Groups 2-4 were each treatment groups that contained 8 animals.

Housing

The hamsters were housed individually in a positive pressure cages (Micro-Vent Environmental System, Allentown Caging and Equipment Co., Allentown, N.J.) with free access to water and to chow (Purina 5000).

Treatment Model

On day (−2), prophylactic gavage was initiated according to the regimen shown in Table 7, below. Animals in all groups were infected on day (−1) by oral gavage with 10⁶ washed cells from an overnight broth of C. difficile (VPI 10463). Animals in all 4 groups were injected subcutaneously with 10 mg of clindamycin phosphate per kg on day 0 (the day following day −1) to induce disease. The hamsters were gavaged in three equal daily doses, on days (−2) to 6, according to the following regimen.

TABLE 7
Treatment Regiment for Hamster C. difficile Challenge Study

| | # of | |
|---|---|---|
| Group | animals | Treatment |
| 1 | 6 | Phosphate buffered saline (sham) |
| 2 | 8 | 1000 mg/kg/day toxin-binding microparticles |
| | | (Y103A2) |
| 3 | 8 | 500 mg/kg/day toxin-binding microparticles |
| | | (Y103A2) |
| 4 | 8 | 250 mg/kg/day toxin-binding microparticles |
| | | (Y103A2) |

The toxin-binding microparticles were administered at 20 mg/mL in phosphate buffered saline. The animals were observed before gavage for morbidity and mortality, as well as the presence or absence of diarrhea on at least a twice-daily basis for 14 days after clindamycin treatment.

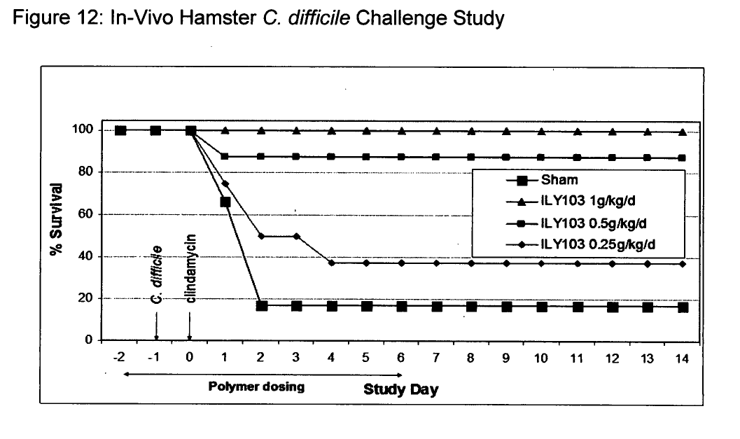

The results, shown in FIG. 12, demonstrate that the toxin-binding microparticles protect hamsters challenged with C. difficile. For the control Group 1 (sham; no toxin-binding microparticles), seven of the animals died on study day two of the fourteen day study. For the treatment Groups 2-4, these data show that survival was dose-dependent and no recurrence was observed. Specifically, for Group 2, none of the hamsters died over the study. For Group 3: one animal died at day 1; two different animals were observed to have wet tail (evidence of diarrhea), but covered fully. For group 4: five animals died; of these, one animal was observed to have wet tail and died within 48 hours of this observation. All other deaths were generally acute (i.e. without prior observation of wet tail).

### Example 8

### Determination of Toxin Binding by ILY103 Nanoparticles

Y103A2 nanoparticles at concentrations ranging from 0.25-5 mg/mL in phosphate buffer solution (PBS) containing 5% fetal bovine serum (Mediatech, Inc., Herndon, Va.) were incubated with purified C. difficile toxin A or B (TechLab, Blacksburg, Va.) at 10 ug/mL for 12-18 hours at 37° C. The mixtures were centrifuged at 10,500×g (Sorvall) at 4° C. for 30 minutes to precipitate complexes of bound toxin with nanoparticles. The amount of free toxin remaining in the supernatant was quantified using the TechLab C. difficile toxin ELISA kit and the percent of toxin bound was calculated. From this data the concentration of nanoparticles that bound 90% of the toxin was calculated. Table 8 lists the screening data from batches of Y103A2.

TABLE 8
Binding Data for Y103A2 Nanoparticles

| Y103A2 | Conc (mg/ml) at | Conc (mg/ml) at | DLS Diameter | DLS |
|---|---|---|---|---|
| Batch ID | 90% ToxA Bound | 90% ToxB Bound | (nm) | PDI |
| TM466D | 2.3 | >5 | | |
| TM473A | 1.8 | >5 | | |
| TM473B | 1.9 | 4.5 | | |
| tilm133 | 2.2 | >5 | 132.8 | 0.099 |
| tilm134 | 1.1 | 2.1 | 126.1 | 0.141 |
| tilm135 | 2 | 3.4 | 233 | 0.141 |
| tilm138 | 5.6 | >5.8 | 433.9 | 0.232 |
| tilm143 | 2.5 | 3.2 | 445.7 | 0.285 |
| tilm144 | 2.4 | 3.5 | 440.6 | 0.319 |
| tilm147B | <1.9 | 3.2 | 232.5 | 0.158 |
| tilm149A | <<1.9 | <1.94 | 223.3 | 0.141 |
| tilm152A | 3.9 | >5 | 205.4 | 0.152 |
| tilm152B | 1.8 | 1.9 | 175.8 | 0.175 |
| tilm155 | 1.8 | 3.3 | 171 | 0.208 |
| tilm158A | 0.93 | 1.9 | 128.8 | 0.204 |
| tilm158B | 1 | 1.5 | 132.1 | 0.216 |
| tilm160A | 1.4 | 1.4 | 127.8 | 0.186 |
| tilm160B | 1.2 | 1.5 | 148.3 | 0.202 |
| tilm164 | 0.93 | 1.9 | 131.8 | 0.222 |
| tilm191A | 0.9 | 2.5 | 102.4 | 0.281 |
| tilm191B | 0.96 | 2.8 | 97.8 | 0.278 |
| tilm196A | 2.1 | 2 | 136.6 | 0.264 |
| tilm196B | 1.7 | 1.8 | 166.4 | 0.262 |
| tilm200 | 3.1 | 4.3 | 235.1 | 0.241 |
| tilm206 | 2.2 | 2.8 | 169.2 | 0.231 |
| tilm208 | 1.1 | 1.5 | 174.9 | 0.248 |
| tilm209A | 0.9 | 1.8 | 137.5 | 0.274 |
| tilm209B | 0.9 | 2.6 | 107.9 | 0.224 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A toxin binding composition comprising a toxin binding moiety and a polymeric particle, the polymeric particle comprising a block copolymer comprising a hydrophilic block and a hydrophobic block, the hydrophobic block being chemically crosslinked or physically enveloped such that the block copolymer forms a micelle in an aqueous medium, the toxin binding moiety being linked to the hydrophilic block.

2. The composition of claim 1 wherein the toxin binding moiety has binding affinity for a bacterial toxin.

3. The composition of claim 1 wherein the toxin binding moiety has binding affinity for a secreted bacterial protein that alters a metabolic process within a mammalian cell.

4. The composition of claim 1 wherein the toxin binding moiety has binding affinity for a secreted bacterial protein that alters a metabolic process within a human cell.

5. The composition of claim 1 wherein the toxin binding moiety binds or neutralizes a toxin that acts on a mucosal surface of a host.

6. The composition of claim 5 wherein the mucosal surface is selected from the group consisting of oral, nasal, respiratory, gastrointestinal, urinary, reproductive and auditory mucosal surfaces.

7. The composition of claim 1 wherein the copolymer can form a micelle in an aqueous medium.

8. The composition of claim 7 wherein the micelle comprises a core and a shell, the core comprising the hydrophobic block and the shell comprising the hydrophilic block.

9. The composition of claim 7 wherein the micelle comprises a polymer block formed from an additional monomer, the additional polymer block chemically crosslinking or physically enveloping the hydrophobic block of the copolymer.

10. The composition of claim 9 wherein the additional polymer block crosslinks or envelopes by polymerizing monomer between the hydrophobic blocks of the block copolymer.

11. The composition of claim 9 wherein the additional monomer is a hydrophobic monomer, a multifunctional monomer, or a combination thereof.

12. The composition of claim 9 wherein the additional monomer is at least one monomer selected from styrene, divinylbenzene, ethylene glycol dimethacrylate, C₁-C₁₂ alcohol esters of acrylic acid, C₁-C₁₂ alcohol esters of methacrylic acid, vinyltoluene, vinylesters of C₂-C₁₂ carboxylic acids, and combinations thereof.

13. The composition of claim 1 wherein the hydrophobic block is a polymer comprising at least one repeat unit selected from C₁-C₁₂ alcohol esters of acrylic acid, C₁-C₁₂ alcohol esters of methacrylic acid, styrene, vinyltoluene, vinylesters of C₂-C₁₂ carboxylic acids, and combinations thereof.

14. The composition of claim 1 wherein the hydrophilic block is a polymer of dimethylacrylamide.

15. The composition of claim 1 wherein the composition has a particle radius from about 75 nm to about 1 micron.

16. A toxin binding composition comprising a toxin binding moiety and a polymeric nanoparticle, the toxin binding moiety being linked to the nanoparticle and the nanoparticle being substantially not absorbed from the gastrointestinal lumen into gastrointestinal mucosal cells.

17. The composition of claim 16 wherein the toxin binding moiety binds a C. difficile toxin.

18. The composition of claim 16 wherein the nanoparticle is a copolymer.

19. The composition of claim 16 wherein the nanoparticle is not a liposome.

20. A toxin binding composition comprising a C. difficile toxin binding moiety and a polymeric particle, wherein at least about 90% of C. difficile toxin A is bound by the composition at a concentration ranging from about 0.1 mg/mL to about 20 mg/mL, the C. difficile toxin A being treated with the toxin binding composition in a phosphate buffer solution containing about 5% fetal bovine serum.

21. The composition-of claim 20 wherein the concentration of the composition needed to bind about 90% of C. difficile toxin A is from about 0.5 mg/mL to about 10 mg/mL.

22. The composition of claim 20 wherein the concentration of the composition needed to bind about 90% of C. difficile toxin A is from about 0.8 mg/mL to about 5 mg/mL.

23. The composition of claim 20 wherein the concentration of the composition needed to bind about 90% of C. difficile toxin A is from about 1 mg/mL to about 3 mg/mL.
